SEQUENCE LISTING (20 sequences)

SEQ ID NO: 1 - RNA, 22 nt, synthetic construct
  misc_feature 1..22: note = Synthetic: cfa-miR-30b-5p
  source 1..22: mol_type = other RNA
tgtaaacatcctacactcagct

SEQ ID NO: 2 - RNA, 24 nt, synthetic construct
  misc_feature 1..24: note = Synthetic: cfa-miR-30d-5p
  source 1..24: mol_type = other RNA
tgtaaacatccccgactggaagct

SEQ ID NO: 3 - RNA, 21 nt, synthetic construct
  misc_feature 1..21: note = Synthetic: cfa-miR-128-3p
  source 1..21: mol_type = other RNA
tcacagtgaaccggtctcttt

SEQ ID NO: 4 - RNA, 22 nt, synthetic construct
  misc_feature 1..22: note = Synthetic: cfa-miR-133a-3p
  source 1..22: mol_type = other RNA
ttggtccccttcaaccagctgt

SEQ ID NO: 5 - RNA, 22 nt, synthetic construct
  misc_feature 1..22: note = Synthetic: cfa-miR-133b-3p
  source 1..22: mol_type = other RNA
tttggtccccttcaaccagcta

SEQ ID NO: 6 - RNA, 21 nt, synthetic construct
  misc_feature 1..21: note = Synthetic: cfa-miR-142-5p
  source 1..21: mol_type = other RNA
cccataaagtagaaagcacta

SEQ ID NO: 7 - RNA, 22 nt, synthetic construct
  misc_feature 1..22: note = Synthetic: cfa-miR-206-3p
  source 1..22: mol_type = other RNA
tggaatgtaaggaagtgtgtgg

SEQ ID NO: 8 - RNA, 22 nt, synthetic construct
  misc_feature 1..22: note = Synthetic: cfa-miR-320-3p
  source 1..22: mol_type = other RNA
aaaagctgggttgagagggcga

SEQ ID NO: 9 - RNA, 23 nt, synthetic construct
  misc_feature 1..23: note = Synthetic: cfa-miR-423a-5p
  source 1..23: mol_type = other RNA
tgaggggcagagagcgagacttt

SEQ ID NO: 10 - RNA, 21 nt, synthetic construct
  misc_feature 1..21: note = Synthetic: cfa-miR-499-5p
  source 1..21: mol_type = other RNA
ttaagacttgcagtgatgttt

SEQ ID NO: 11 - RNA, 22 nt, synthetic construct
  misc_feature 1..22: note = Synthetic: cfa-let-7b-5p
  source 1..22: mol_type = other RNA
tgaggtagtaggttgtgtggtt

SEQ ID NO: 12 - RNA, 22 nt, synthetic construct
  misc_feature 1..22: note = Synthetic: cfa-let-7e-5p
  source 1..22: mol_type = other RNA
tgaggtaggaggttgtatagtt

SEQ ID NO: 13 - RNA, 22 nt, synthetic construct
  misc_feature 1..22: note = Synthetic: cfa-let-7i-5p
  source 1..22: mol_type = other RNA
tgaggtagtagtttgtgctgtt

SEQ ID NO: 14 - RNA, 22 nt, synthetic construct
  misc_feature 1..22: note = Synthetic: cfa-miR-29a-3p
  source 1..22: mol_type = other RNA
tagcaccatctgaaatcggtta

SEQ ID NO: 15 - RNA, 22 nt, synthetic construct
  misc_feature 1..22: note = Synthetic: cfa-miR-486-5p
  source 1..22: mol_type = other RNA
tcctgtactgagctgccccgag

SEQ ID NO: 16 - RNA, 23 nt, synthetic construct
  misc_feature 1..23: note = Synthetic: cfa-miR-17-5p
  source 1..23: mol_type = other RNA
caaagtgcttacagtgcaggtag

SEQ ID NO: 17 - RNA, 22 nt, synthetic construct
  misc_feature 1..22: note = Synthetic: cfa-miR-130b-3p
  source 1..22: mol_type = other RNA
cagtgcaatgatgaaagggcat

SEQ ID NO: 18 - RNA, 23 nt, synthetic construct
  misc_feature 1..23: note = Synthetic: cfa-miR-20a-5p
  source 1..23: mol_type = other RNA
taaagtgcttatagtgcaggtag

SEQ ID NO: 19 - RNA, 19 nt, synthetic construct
  misc_feature 1..19: note = Synthetic: cfa-miR-23a-3p
  source 1..19: mol_type = other RNA
atcacattgccagggattt

SEQ ID NO: 20 - RNA, 22 nt, synthetic construct
  misc_feature 1..22: note = Synthetic: cfa-miR-26a-5p
  source 1..22: mol_type = other RNA
ttcaagtaatccaggataggct

(19) United States
(12) Patent Application Publication
(10) Pub. No.: US 2025/0305048 A1
(43) Pub. Date: Oct. 2, 2025

(54) ASSESSMENT AND DIFFERENTIAL DIAGNOSIS OF CARDIOVASCULAR DISEASE IN COMPANION ANIMALS USING A MICRORNA ASSAY

(71) Applicant: MI:RNA Ltd., Edinburgh (GB)

(72) Inventors: Eve HANKS, Edinburgh (GB); Robert COULTOUS, Whitburn (GB)

(73) Assignee: MI:RNA Ltd., Edinburgh (GB)

(21) Appl. No.: 19/043,498

(22) Filed: Feb. 2, 2025

Related U.S. Application Data
(63) Continuation-in-part of application No. PCT/IB2024/000461, filed on Aug. 28, 2024.
(60) Provisional application No. 63/579,089, filed on Aug. 28, 2023.
(60) Provisional application No. 63/605,127, filed on Dec. 1, 2023.
(60) Provisional application No. 63/549,256, filed on Feb. 2, 2024.
(60) Provisional application No. 63/566,582, filed on Mar. 18, 2024.
(60) Provisional application No. 63/662,743, filed on Jun. 21, 2024.

(51) Int. Cl. C12Q 1/6883; G16B 25/10; G16B 40/20

(52) U.S. Cl. CPC C12Q 1/6883; G16B 25/10; G16B 40/20

(57) ABSTRACT

A method of assessing expression profiles of miRNA markers using predictive classification models to distinguish between non-diseased and diseased mitral valve disease, non-diseased and diseased DCM, non-diseased and diseased HCM. Additionally, an assessment of the same method is provided to discriminate pre-clinical from clinical MMVD or DCM patients. Also provided is a method of differentially diagnosing MMVD patients from DCM patients or from healthy controls.

### REFERENCE TO RELATED APPLICATIONS

This application is a Continuation-In-Part application of International Patent Application No. PCT/IB2024/000461, filed Aug. 28, 2024, which claims the benefit of priority to U.S. Provisional Application No. 63/579,089, filed Aug. 28, 2023, and to U.S. Provisional Application No. 63/605,127, filed Dec. 1, 2023, and to U.S. Provisional Application No. 63/566,582, filed Mar. 18, 2024, and to U.S. Provisional Application No. 63/662,743, filed Jun. 21, 2024. This application also claims the benefit of priority to U.S. Provisional Application No. 63/549,256, filed Feb. 2, 2024. All of the above applications are hereby incorporated by reference in their entireties.

### SEQUENCE LISTING

The application contains a Sequence Listing which has been submitted electronically in .XML format and is hereby incorporated by reference in its entirety. Said. XML copy, created on May 1, 2025, is named “068075.005CIP.xml” and is 26,553 bytes in size. The sequence listing contained in this. XML file is part of the specification and is hereby incorporated by reference herein in its entirety.

### FIELD

The present invention relates to isolated nucleic acid molecules known as microRNAs (miRNAs) and miRNA precursor molecules and their use in diagnosis and therapy. The invention also relates to a method and a kit for assessing and differentially diagnosing cardiovascular disease in a subject. The invention further relates to methods for assessing and differentially diagnosing between a healthy subject and a diseased subject having myxomatous mitral valve disease (MMVD), mitral regurgitation (MR), dilated cardiomyopathy disease, hypertrophic cardiomyopathy (HCM). Additionally, the invention provides methods for differentiating between the diseases.

### BACKGROUND

MMVD is the most common cardiovascular disease in dogs (Borgarelli M, et al., J Vet Car-diol. 2004; 6:27-34; Borgarelli M, Häggström J. Vet Clin North Am Small Anim Pract. 2010; 40:651-663.). Progressive degenerative lesions of the mitral valve lead to mitral regurgitation and a gradually increasing left sided volume load. Increasing left sided filling pressures eventually lead to left sided congestive heart failure (CHF) (Häggström J, Höglund K, Borgarelli M., J Small Anim Pract. 2009; 5:25-33.). MMVD can be graded in 4 stages: stage A, healthy dogs at risk for developing MMVD; stage B, dogs with evidence of mitral valve regurgitation and no clinical signs of CHF; stage C, dogs with clinical signs of CHF; or stage D, dogs with clinical signs of CHF refractory to treatment (Keene, B W, et al., J Vet Intern Med. 2019; 33:1127 1140). Stage B, also known as the pre-clinical period, is further divided into stage B1 for patients with no significant remodelling changes, and stage B2 when echocardiographic evidence shows left atrial enlargement and significant remodelling (Keene, B W, et al., J Vet Intern Med., supra). The prevalence of MMVD is higher in smaller dogs (<20 kg) and increases markedly with age, with up to 85% of dogs with valve lesions by the age of 13 Borgarelli M, et al., J Vet Car, supra; Keene, B W, et al., J Vet Intern Med., supra).

Serial echocardiographic examination is recommended as the most sensitive method of monitoring MMVD (Hezzell M J, et al., J Vet Cardiol. 2012; 14:269-279), but is rarely possible or practical in a clinical setting. The detection of cardiac biomarkers (CBs), such as N-terminal pro-brain natriuretic peptide (NT-proBNP) and cardiac troponin I (cTnI) can have clinical use in the confirmation or staging of clinical MMVD in dogs (Eriksson, A. S., et al., Am. J. Vet. Res., 62:1818-1824 (2001); Ferasin, L., et al., J. Vet. Intern. Med., 27:286-292 (2013); Maeda, K., et al., J. Appl. Physiol. (1985) 89:458-464 (2000); Magga, J., et al., Ann. Med. 30 Suppl 1:39-45 (1998)), but not for the detection of pre-clinical MMVD (Porter A., Rozanski E., et al., 2016, Can. Vet. 57:641-645.). NT-proBNP is released into the blood circulation in response to myocardial wall stretch and cTnI following cardiomyocyte injury. Early therapeutic intervention in pre-clinical MMVD patients (stage B2) has proven useful in increasing the preclinical period by up to 15 months (Boswood A, et al., J Vet Intern Med. 2016 November; 30 (6): 1765-1779), but reliable association between CBs and stage of MMVD are lacking.

Mitral regurgitation (MR) is a pathological heart murmur commonly associated with reduced performance in horses. It occurs due to a leak in the mitral valve and can lead to increases in left-atrial pressure and dilation, potentially leading to atrial fibrillation, pulmonary hypertension, sudden death, and congestive heart failure.

Consequently, there is a need for reliable tools to diagnosis and stage MMVD and MR in dogs and horses. A promising approach is the application of microRNA (miRNA) profiling. MicroRNAs are small, non-coding RNA molecules that regulate gene expression. Found in tissue (e.g., heart valves) (Yang, V. K., et al. (2018) PLOS One 13 (1): e0188617.), within exosomes (microvesicles) or circulating cell-free in plasma (Yang, V. K., et al. (2017), J Extracell Vesicles 6 (1): 1350088), they have received increasing recognition for their potential role in veterinary cardiology (Reis-Ferreira, A., et al. (2022), Vet Sci 9 (10)). Linked to numerous biological processes, they can be altered in various pathophysiological processes, making them ideal biomarkers. However, limitations in their use as CBs exist as they have no units of measurement, and a plethora of different miRNAs are linked to MMVD in dogs (Bagardi, M., et al. (2022), PLOS One 17 (7): e0266208; Ghilardi, S., et al. (2022), PLOS One 17 (12): e0274724; Hulanicka, M., et al. (2014), BMC Vet Res 10:205; Jung, S. and A. Bohan (2018), Am J Vet Res 79 (2): 163-169; Li, Q., et al. (2015), Int J Mol Sci 16 (6): 14098-14108), indicates that no single marker can be used as a gold standard in isolation.

Hypertrophic cardiomyopathy (HCM) is the most common heart disease in cats and the most common cause of heart failure in this species, affecting as many as one in seven (the vast majority of cases are subclinical) (Riesen et al., 2007; Rush et al., 1998). HCM occurs primarily in domestic cats and rarely in small dogs. It has also been reported in cattle. HCM is rare in dogs when compared to cats and humans with fewer than 30 cases documented in either single case reports (Marks C A. J Am Vet Med Assoc. 1993; 203:1020-1022; Thomas W P, et al. J Am Anim Hosp Assoc. 1984; 20:253-260; Pang D, et al. Vet J. 2005; 46:1122-1125; Washizu M, et al. J Vet Med Sci. 2003 65:753-756; Yamada E. J Jpn Vet Med Assoc. 1983; 36:12-16). This disease is characterized by an abnormal thickening (hypertrophy) of one or several areas of the walls of the heart, usually of the left ventricle. While a genetic mutation of one or more of the sarcomeric proteins has been proposed to be the cause of HCM in most cats, a specific mutation has only been identified for Maine coon and Ragdoll cats (Meurs et al., 2005 and 2007; Kittleson et al., 1999). In most cats identified to have HCM, the heart disease is the eventual cause for death. HCM together with restrictive cardiomyopathy (RCM) are classified as diasystolic dysfunctions.

Dilated Cardiomyopathy (DCM) is a canine heart condition characterised by enlargement and weakening of the heart muscle, leading to reduced cardiac function and arrhythmia. It typically develops in canines >10 kg. DCM often leads to cardiac remodelling, with the heart chambers becoming larger and less efficient at pumping blood. As such, the miRNA panel designed to detect cardiac remodelling in canine myxomatous mitral valve disease (MMVD) may also not only be suitable for the diagnosis of DCM, but have the capacity to differentiate DCM from HCM cases.

Therefore, disclosed herein are compositions and methods for assessment and diagnosis of MMVD, MR, DCM and HCM in a subject by analysing the expression pattern of 15 miRNAs markers by predictive classification models using the miRNA markers as a miRNA panel to discriminate diseased patients from healthy controls. Also disclosed are methods of using the miRNA panel for the assessment of the same method to discriminate pre-clinical (stage B) from clinical (stage C/D) MMVD patients. Additionally, disclosed herein is the use of the miRNA panel for the diagnosis of DCM, and the differential diagnosis of DCM from HCM cases.

### SUMMARY

In accordance with the purpose(s) of this invention, as embodied and broadly described herein, this invention, in one aspect, relates to a method in a computer-implemented system comprising at least one processor and at least one memory, the at least one memory comprising instructions executed by the at least one processor to cause the at least one processor to implement one or more predictive classification models to assess and differentially diagnose a cardiac disease or conditions in a subject, comprising the steps of: (a) obtaining a sample from the subject; (b) determining a level of expression of each of a plurality of miRNA molecules within the sample; (c) applying the one or more predictive classification models to the expression of each of a plurality of miRNA molecules; (d) using the predictive classification models to differentially classify the diseased state of the cardiac disease or condition in the subject; and (e) using the classification of the diseased state of the cardiac disease or condition to predict the disease condition of the subject, wherein the cardiac condition is myxomatous mitral valve disease (MMVD), mitral regurgitation (MR), dilated cardiomyopathy disease (DCM), or hypertrophic cardiomyopathy (HCM). The method further comprises a step of using one or more machine learning algorithms to generate predictive classification models.

In one embodiment, the one or more predictive classification models compares the level of expression of each miRNA molecule with at least one pre-determined reference level characteristic of a non-diseased subject for each one of the plurality of the miRNA molecules of step (b), wherein a deviation of the level of expression of said miRNA molecules from step (b) in comparison with the at least one reference level allows for the diagnosis and/or prognosis of the disease. The plurality of miRNA molecules from a panel selected from a group consisting of miRNAs having at least 99% sequence identity to SEQ ID NO: 1, 2, 3, 4, 5, 6, 7, 8, 9, 10, 11, 12, 13, 14, 15 or the combination thereof.

In another embodiment, the application of the predictive classification models distinguishes non-diseased subjects from diseased subjects with the cardiac diseases or conditions. The non-diseased subjects correspond to stage A subjects as classified by the American College of Veterinary Internal Medicine (ACVIM) classification system and the diseased subjects with mitral valve diseases or conditions correspond to stages B1, B2, C and D subjects as classified by the ACVIM classification system.

In yet another embodiment, the application of the predictive classification models distinguishes pre-clinical mitral valve disease or condition subjects from clinical mitral valve disease or condition subject. The preclinical mitral valve disease or condition subjects correspond to stage B1 and stage B2 subjects as classified by the ACVIM classification system and the clinical mitral valve disease or condition subjects correspond to stage C and stage D subjects as classified by the ACVIM classification system.

In one embodiment, the mitral valve disease or condition is myxomatous mitral valve disease (MMVD) or mitral regurgitation (MR).

In yet another embodiment, the application of the predictive classification models distinguishes non-diseased subjects from diseased subjects with dilated cardiomyopathy or conditions. In another embodiment, application of the predictive classification models distinguishes non-diseased subjects from diseased subjects with the hypertrophic cardiomyopathy (HCM) disease or condition.

In one embodiment, the method differentially assesses and diagnoses one cardiac disease from another. In specific embodiments, the method differentially assesses and diagnoses myxomatous mitral valve disease (MMVD) from dilated cardiomyopathy (DCM).

In one other embodiment, the subject is a mammal. The mammal is selected from a group of non-human mammals consisting of dogs, cast, and horses.

In another embodiment, the method further comprises the step of using one or more machine learning algorithms to generate predictive classification models. The method also comprises the use of a combination of predictive classification models.

In yet another embodiment, the method further comprises the use of at least one normalizer and/or control miRNA molecule. The control miRNA molecule is an off-species control miRNA molecule. The at least one normalizer is selected from a group consisting of miRNAs having at least 99% sequence identity to SEQ ID NO: 16, 17, 18, 19, and 20.

In one other embodiment, wherein the sample is selected from a group consisting of a tissue or organ sample, blood sample, urine, saliva, milk and cerebrospinal fluid sample. The blood sample is selected from the group consisting of serum, plasma, cell-free blood, whole blood and its components, blood derived products or preparations thereof. The miRNAs are cell free miRNAs.

In another aspect, the invention relates to a kit for use in performing the method of differentially assessing and diagnosing a diseased state of the mitral valve disease or condition or dilated cardiomyopathy disease or conditions in a subject comprising means for determining the level of expression of miRNA molecules having at least 99% sequence identity to SEQ ID NO: 1, 2, 3, 4, 5, 6, 7, 8, 9, 10, 11, 12, 13, 14, 15 or the combination thereof.

In yet another aspect, the invention relates to a method of selecting a panel for use in disease diagnosis comprising the steps of: (a) obtaining a sample from the subject; (b) determining a level of expression of each of a plurality of miRNA molecules within the sample, having at least 99% sequence identity to SEQ ID NO: 1, 2, 3, 4, 5, 6, 7, 8, 9, 10, 11, 12, 13, 14, and 15; (c) using a computer-implemented system comprising at least one processor and at least one memory, the at least one memory comprising instructions executed by the at least one processor to cause the at least one processor to apply machine learning algorithms to generate one or more predictive classification models; (d) applying the one or more predictive classification models to the expression of each of a plurality of miRNA molecules; and (e) using the predictive classification models to diagnose a cardiac disease in the subject; wherein the cardiac condition is myxomatous mitral valve disease (MMVD), mitral regurgitation (MR), dilated cardiomyopathy disease, or hypertrophic cardiomyopathy (HCM). In other embodiments the predictive classification models are used to differentially assess and diagnose a preclinical or clinical stage of the cardiac disease or differentially diagnoses one cardiac disease from another.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 shows a flow diagram describing the clinical cohort recruited to the study.

FIG. 2 is a heatmap representation of 15 miRNA expression profiles in 97 dog blood samples divided into two groups according to their disease status, healthy controls or MMVD patients. MicroRNA expression is represented as a colour gradient based on mean fluorescence, increasing from bottom to top. Samples and miRNA identification numbers are shown in the right-hand side Y-axis and top X-axis, respectively.

FIGS. 3A-3D show a cross-validated diagnostic performance of predictive model to distinguish healthy controls from MMVD cases. FIG. 3A shows a principal component analysis (PCA) biplot of processed miRNA profiles of all 97 collected samples that distinguish healthy controls from MMVD dogs. FIG. 3B shows a cross-validated receiver operating curve (ROC) for healthy controls versus MMVD dogs (combining Stage B1/B2 and Stage C/D MMVD) is shown. Area under the curve (AUC) and confidence interval (95% CI) are indicated at the bottom right of the diagram. FIG. 3C shows a summary of cross-validated performance statistics from predictive model to distinguish healthy controls from MMVD dogs (95% CI showed in parenthesis where available). FIG. 3D shows predicted status probabilities for each collected sample. Samples are ordered from left to right on the X-axis according to their probability. The classification is based on a usual 0.5 probability threshold (dotted line), background colour indicates actual dog disease status. The misclassified samples are indicated at the bottom of the diagram by green and orange dots in accordance with their disease status.

FIG. 4 shows a flow diagram further describing the MMVD clinical cohort recruited to the study.

FIG. 5 is a microRNA expression profile heatmap of stage B1/B2 and stage C/D MMVD cases. Heatmap representation of 15 miRNA expression profiles in 47 dog blood samples classified into two groups according to their MMVD disease stage: stage B1/B2 and stage C/D. Classification is based on clinical and cardiac exam, following ACVIM guidelines. MicroRNA expression is represented as a colour gradient based on mean fluorescence, increasing from bottom to top. Samples and miRNA identification numbers are shown in the right-hand side Y-axis and top X-axis, respectively.

FIGS. 6A-D show the Cross-validated diagnostic performance of predictive model to distinguish Stage B1/B2 from Stage C/D MMVD dogs. FIG. 6A shows a principal component analysis (PCA) biplot of processed miRNA profiles of 47 collected samples that distinguish Stage B1/B2 from Stage C/D MMVD dogs. FIG. 6B shows a cross-validated receiver operating curve (ROC) for Stage B1/B2 MMVD dogs versus Stage C/D MMVD dogs is shown. Area under the curve (AUC) and confidence interval (95% CI) are indicated at the bottom right of the diagram. FIG. 6C shows summary cross-validated performance statistics from the predictive model to distinguish Stage B1/B2 from Stage C/D MMVD dogs (95% CI showed in parenthesis where available). FIG. 6D shows the predicted status probabilities for each collected sample. Samples are ordered from left to right on the X-axis according to their probability. The classification is based on a usual 0.5 probability threshold (dotted line), background colour indicates actual dog disease status. The misclassified samples are indicated at the bottom of the diagram by green and orange dots in accordance with their disease status.

FIG. 7 shows the initial analysis of MR test data.

FIG. 8 shows accuracy metrics for various machine learning models for MR test data.

FIGS. 9A-9B show PCA biplots of training data set with miRNA profiles distinguished by diagnosis group: DCM and MMVD (FIG. 9A) and DCM, MMVD and Control (FIG. 9B). The miRNA profiles and individual miRNA markers are represented by points and rays respectively.

FIGS. 10A-10C show KNN classifier results for DCM against health control animals: (FIG. 10A) receiver operating characteristic curve, (FIG. 10B) approximated 2D prediction grid and actual miRNA profile group, (FIG. 10C) predictor contribution to identify DCM disease status (relative to reference miRNA panel).

FIGS. 11A-11C show KNN classifier results for combined DCM-MMVD diseased against health control animals: (FIG. 11A) receiver operating characteristic curve, (FIG. 11B) approximated 2D prediction grid and actual miRNA profile group, (FIG. 11C) predictor contribution to identify disease status (relative to reference miRNA panel).

FIGS. 12A-12B shows the AUC (FIG. 12A) and a UMAP (FIG. 12B) demonstrating distinct separate grouping of the DCM and MMVD miRNA profiles.

FIGS. 13A-13B show the initial analysis of HCM test data.

FIGS. 14A-14B shows accuracy metrics of various learning models for HCM.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The terms “comprise(s),” “include(s),” “having,” “has,” “can,” ““contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk and cerebrospinal fluid sample.

As used herein, the term “blood sample” refers to serum, plasma, cell-free blood, whole blood and its components, blood derived products or preparations. Plasma and serum are very useful as shown in the examples.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNA. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample. The control may include a sample obtained from a healthy or non-diseased subject or a subject, which is not at risk of or suffering from MMVD. Reference level specifically refers to the level of miRNA or miRNA expression quantified in a sample from a healthy subject, from a subject, which is not at risk of or suffering from MMVD. Specifically a more than 1.3, 1.4, 1.5, 1.6, 1.7, 1.8, 1.9 or 2.0 fold difference between the reference level of one or more miRNAs as defined herein obtained from a sample of a subject. Additionally, a control may also be a standard reference value or range of values, i.e. such as stable expressed miRNAs in the samples, for example the endogenous control.

“Animal(s)”, as used herein, unless otherwise indicated, refers to an individual animal that is a mammal. Specifically, mammal refers to a vertebrate animal that is human and non-human, which are members of the taxonomic class Mammalia. Non-exclusive examples of non-human mammals include companion animals. Non-exclusive examples of a companion animal include: dog, cat, and horse, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can further refer to wild animals, including, but not limited to bison, elk, deer, venison, duck, fowl, fish, and the like.

The term “companion animals” refers to domesticated animals living in the same quarters as humans. Companion animals, commonly referred to as pets, includes dogs, cats, horses, birds, rabbits, goats, and gerbils. In particular embodiments, the companion animal is a dog or a cat, including all breeds thereof.

As used herein, the terms “dog” or “canine” are used interchangeably and refer to any member of the Canidae family including, but not limited to, Canis lupus, Canisfamiliaris, Canis latrans, Canis dingo, Lycaon pictus, Chrysocyon brachyurus, Atelocynus microis, Cuon alpinus, Speothos venaticus, Nyctereutes procyonoides, Vulpes vulpes, and Alopex lagopus. In certain embodiments, the dog or canine is Canisfamiliaris.

As used herein, the terms “cardiac dysfunction,” “cardiovascular disease” or “cardiac disease” mean any diseases, disorders, or conditions of the heart, including age-related diseases, disorders, or conditions related to the heart. In some embodiments, cardiac dysfunction includes cardiomyopathy, such as hypertrophic cardiomyopathy (HCM) or dilated cardiomyopathy (DCM); valve disease, such as mitral valve disease (MVD); mitrial valve (MR) disease and cardiac hypertrophy, such as pressure-overload hypertrophy.

“Asymptomatic (occult, preclinical) heart failure” as used herein, unless otherwise indicated, refers to any contractile disorder or disease of the heart which is due to MMVD.

“Congestive heart failure”, or “heart failure” unless otherwise indicated, refers to a manifested process wherein the heart is unable to keep up with the demands of blood supply to the body and generally results in fluid buildup in the lungs resulting from increased cardiac and pulmonary pressures. The term(s) also relate to any contractile disorder or disease of the heart. Clinical manifestations are as a rule the result of changes to the heart's cellular and molecular components and to mediators that drive homeostatic control that leads to an increase in heart size and deterioration of cardiac function.

“Myxomatous mitral valve degeneration (MMVD)”, unless otherwise indicated, refers to the manifested process of mitral valve degeneration. MMVD is generally detected as a heart murmur by auscultation. MMVD also includes synonymous medicinal terms: mitral valve disease (MVD); degenerative mitral valve disease (DMVD); chronic valve disease (CVD); chronic valvular heart disease (CVHD); and atrial ventricular valvular insufficiency (AVVI).

As used herein, a “biological sample” includes samples from biological fluids and tissues. Biological fluids include whole blood, blood plasma, blood serum, sputum, urine, saliva, milk, sweat, lymph, cerebrospinal fluid, and alveolar lavage. Tissue samples include biopsies from solid lung tissue or other solid tissues, lymph node biopsy tissues, biopsies of metastatic foci. Methods of obtaining physiological samples are well known.

As used herein, the terms “marker”, “biomarker” (or fragment thereof) and their synonyms, which are used interchangeably, refer to molecules that can be evaluated in a sample and are associated with a physical condition. For example, markers include expressed genes or their products (e.g., proteins) or autoantibodies to those proteins that can be detected from human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The term “biomarker” can also refer to a portion of a polypeptide (parent) sequence that comprises at least 5 consecutive amino acid residues, preferably at least 10 consecutive amino acid residues, more preferably at least 15 consecutive amino acid residues, and retains a biological activity and/or some functional characteristics of the parent polypeptide, e.g. antigenicity or structural domain characteristics. The present markers described herein is one or more mRNAs. In particular embodiments, the present markers are a panel of one or more mRNAs.

As used herein, the term “classifier model” may be used interchangeably with the terms “predictive classifier model,” “predictive classifying model,” “predictive classification model” or the like. It may also understood in the present methods that use of the markers in a panel may each contribute equally in the classifier model or certain biomarkers may be weighted wherein the markers in a panel contribute a different weight or amount in the classifier model. Biomarker may include any biological substance indicative of the presence of cardiac disease, including but not limited to, genetic, epigenetic, proteomic, glycomic or imaging biomarkers. Biomarkers include molecules secreted by biological or blood samples, including cell-free DNA, mRNA, and protein-based products, etc.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming. Statistical modeling relies on finding relationships between variables (e.g., mathematical equations) to predict an outcome.

As used herein the term, “Receiver Operating Characteristic Curve,” or, “ROC curve,” is a plot of the performance of a particular feature for distinguishing two populations, diseased subjects or patients, and controls, e.g., those without disease. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied, weighted, etc.) to provide a single combined value which can be plotted in a ROC curve. The ROC curve is a plot of the true positive rate (sensitivity) of a test against the false positive rate (1-specificity) of the test. ROC curves provide another means to quickly screen a data set. As used herein, performance of the present classifier models is determined using computed ROC curves with sensitivity and specificity values. The performance is used to compare models, and also importantly, to compare models with different variables to select a classifier model with the highest accuracy as to predicting having or developing cancer, for a patient.

In other embodiments, the choice of the markers may be based on the understanding that each marker, when measured and normalized, contributed unequally as an input variable for the classifier model.

In still other embodiments, a machine learning system may analyze values from biomarker panels without normalization of the values. Thus, the raw value obtained from the instrumentation to make the measurement may be analyzed directly.

A blood sample from the subject, is obtained and sent to a laboratory qualified to test the sample using a panel of biomarkers, such as those used to train the present classifier models generated by a machine learning system. Non-limiting lists of such biomarkers are herein included throughout the specification including the examples. In lieu of blood, other suitable bodily fluids such a sputum or saliva might also be utilized.

The measured values of the biomarkers are then used as input values, along with weight, to be used with the classifier model in a computer implemented system. An output value is obtained and compared to a threshold value wherein the threshold is empirically determined and set to separate patients in a cardiac disease from those without a cardiac disease. The threshold value is empirically determined using longitudinal clinical data. If the risk calculation is to be made at the point of care, rather than at the laboratory, a software application compatible with mobile devices (e.g. a tablet or smart phone) may be employed.

For those subjects assessed to have a cardiac disease, the input variables of measured biomarkers may be used with the classifier model in a computer implemented system to differentially diagnosis the stages of the cardiac disease or specific cardiac diseases. An output value is obtained and compared to the longitudinal clinical data used to train the classifier model and assigned a disease stage. In certain embodiments, the disease is further defined by a specific cancer type, e.g. lung cancer.

### II. Compositions

The present invention provides genomic identifiers for MMVD. These can be used as target nucleic acid sequences for diagnosis of MMVD in a subject. The diagnostic targets can be used for identification of MMVD in a sample.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

Provided herein is a method for assessing and diagnosing MMVD in a subject, comprising the steps of: (a) determining the level of expression of each of a plurality of miRNAs within a sample from a subject; and (b) using one or more Artificial Intelligence (AI) model to predict the disease condition of the subject.

A. miRNAs

Nucleotide sequences of mature miRNAs and their respective precursors are known in the art and available from the database miRBase or from Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide comprising or consisting of the nucleotide sequence of any one of SEQ ID NOs: 1-20.

Furthermore, identical polynucleotides as used herein in the context of a polynucleotide to be detected by the method as described herein may have a nucleic acid sequence with an identity of at least 90%, 95%, 97%, 98% or 99% to a polynucleotide comprising or consisting of the nucleotide sequence of any one of SEQ ID NOs: 1-20 including one, two, three or more nucleotides of the corresponding pre-miRNA sequence at the 5′end and/or the 3′end of the respective seed sequence.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and also its isoforms and variants.

The plurality of miRNAs from a panel comprising the following: cfa-miR-30d-5p, cfa-miR-128-3p, cfa-miR-133a-3p, cfa-miR-133b-3p, cfa-miR-142-5p, cfa-miR-206-3p, cfa-miR-320-3p, cfa-miR-423a-5p, cfa-miR-499-5p, cfa-let-7b-5p, cfa-let-7e-5p, cfa-let-7i-5p, cfa-miR-29a-3p and cfa-miR-486-5p having with at least 85%, 90%, 95% or 99% sequence identity to SEQ ID NO: 1, 2, 3, 4, 5, 6, 7, 8, 9, 10, 11, 12, 13, 14, 15. The names of the miRNA molecules and associated sequences that are used in the method of the invention are set out below in Table 1.

The method further comprises the use of at least one normalizer and/or an off-species control miRNA molecule. At least one normalizer is used to ‘normalize’ data, i.e. to control for variation between the samples tested in the method of the invention, and the at least one control is used to try to ensure there are no failure or false readings in the results. An off-species control is added in to show that the miRNAs detected are relevant to the species panel. The off-species control is an miRNA from another species, i.e. not a ruminant. Advantageously, the use of an off-species controls provides another layer of control to distinguish between background or non-specific signals and a positive result. The sequences of the normalizers and the off-species controls that were used are provided below in Table 2.

It is preferred that the method comprises the step of assessing the relative levels of miRNA expression of each one of miRNA molecules cfa-miR-30d-5p, cfa-miR-128-3p, cfa-miR-133a-3p, cfa-miR-133b-3p, cfa-miR-142-5p, cfa-miR-206-3p, cfa-miR-320-3p, cfa-miR-423a-5p, cfa-miR-499-5p, cfa-let-7b-5p, cfa-let-7e-5p, cfa-let-7i-5p, cfa-miR-29a-3p and cfa-miR-486-5p within a sample from a subject and using the data obtained from measurement of the expression levels to determine the presence or absence of disease in a subject.

The plurality of target miRNAs form a panel comprising the following: cfa-miR-30d-5p, cfa-miR-128-3p, cfa-miR-133a-3p, cfa-miR-133b-3p, cfa-miR-142-5p, cfa-miR-206-3p, cfa-miR-320-3p, cfa-miR-423a-5p, cfa-miR-499-5p, cfa-let-7b-5p, cfa-let-7e-5p, cfa-let-7i-5p, cfa-miR-29a-3p and cfa-miR-486-5p.

### III. Methods of Detection

### A. Cardiac Disease

Presented herein are methods of assessing and differentially diagnosing cardiac diseases in a subject using a miRNAs panel of biomarkers. In certain embodiments, a cardiac disease is cardiac fibrosis, cardiac enlargement, cardiac hypertrophy, cardiac dilation, hypertrophic cardiomyopathy, heart failure, post-myocardial infarction remodeling, myocardial infarction, cardiomyopathy (for example, hypertrophic cardiomyopathy, restrictive cardiomyopathy, dilated cardiomyopathy (DCM), idiopathic dilated cardiomyopathy, or dilated cardiomyopathy with arrhythmias), diastolic heart failure, chronic atrial fibrillation, primary pulmonary hypertension, acute respiratory distress syndrome, brugada syndrome, progressive cardiac conduction disease, uremic pericarditis, anthracycline cardiomyopathy, arterial fibrosis, post-radiation lymphatic fibrosis, sarcoidosis, scleroderma, endocardial fibroelastosis, serotonergic excess, cardiac valvulopathy, atrial fibrosis, atrial fibrillation, mitral valvular disease, hypertension, chronic ventricular dysfunction, pressure and volume overload, or myocardial fibrosis.

Cardiac disease in dogs takes a number of different forms, with the most common cardiac diseases being the acquired diseases, myxomatous mitral valve degeneration (MMVD) and canine dilated cardiomyopathy (DCM). Treatment for the heart diseases MMVD and DCM depend the dog's current clinical stage, with treatment during the earlier stages providing optimal outcomes. However, clinical signs of MMVD and DCM are similar because the left side of the heart is primarily affected. Therefore, it is important to not only provide differential diagnosis for the clinical stages of MMVD and DCM, it is also important to provide differential diagnosis between MMVD and DCM. Provided herein are methods of selecting a miRNA panel for use in MMVD or DCM disease diagnosis. Also provided herein are methods for differential diagnosis of the clinical states of MMVD and/or DCM. Additionally, methods for differential diagnosis between MMVD and DCM subjects are provided herein.

### B. Myxomatous Mitral Valve Degeneration MMVD

In one embodiment, the invention provides methods for assessing and diagnosing myxomatous mitral valve degeneration (MMVD). MMVD is the most common acquired type of heart disease and new heart murmurs in older dogs. A heart murmur is a sound heard with every heartbeat and is caused by turbulent blood flow in the heart. MMVD is a manifestation of a process that can affect the mitral valve. MMVD affects primarily small breed dogs later in life but can affect larger breed dogs. Some smaller breed dogs are affected earlier in life than others with the Cavalier King Charles Spaniel being the most prominent breed described to date.

The mitral valve is the valve between the left atrium and the left ventricle. Oxygenated blood from the lungs enters the left atrium, passes through the mitral valve into the left ventricle and subsequently pumped to the body. The mitral valve closes when the left ventricle contracts which prevents blood from flowing back into the left atrium. A healthy mitral valve is thin and supple and is anchored in place by chordae tendonae (CT). Myxomatous degeneration is a process that occurs when the valve becomes thickened with formation of small nodules which prevent complete closing of the valves allowing back flow (mitral regurgitation) of blood into the left atrium. Over time, the atrium and ventricles compensate by enlarging and the leak progressively worsens. As leaking volume increases, atrial pressure increases. In some instances, CT may rupture causing a partially unanchored mitral valve (mitral valve pro-lapse). The increase in pressure is transmitted to the lungs leading to CHF.

A heart murmur is generally the earliest means by which MMVD can be detected. After the murmur is detected, MMVD symptoms may not appear for three to four years. Often the first outward sign of worsening MMVD is a cough or increased respiratory effort which may be due to airway pressure from the enlarged heart and/or fluid congestion in the lungs and heart.

Heart failure is divided in different stages, which were defined by different classification systems, e.g. the International Small Animal Cardiac Health Council (ISACHC), the New York Heart Association (NYHA) functional classification systems and the currently used classification according to the Consensus Statements of the American College of Veterinary Internal Medicine (ACVIM), 2009. To remove any ambiguity between classification systems, the classification systems described below are to be considered synonymous.

In another embodiment, the invention presented herein provides methods for differentially diagnosing stages of MMVD. Classification according to the International Small Animal Cardiac Health Council (ISACHC) System: Class I: asymptomatic (also known as occult or preclinical); Class IA: no evidence of compensation for underlying heart disease (no volume overload or pressure overload detected radiographically or echocardiographically); Class IB: clinical signs of compensation for underlying heart disease (volume overload or pressure overload detected radiographically or echocardiographically); Class II: mild to moderate heart failure with clinical signs at rest or with mild exercise (treatment required); Class III: advanced heart failure; clinical signs of severe congestive heart failure; Class IIIA: home treatment possible; and Class IIIB: requires hospitalization.

New York Heart Association (NYHA) functional classification system: Class I: describes patients with asymptomatic heart disease (e.g., chronic valvular heart disease (CVHD) is present, but no clinical signs are evident even with exercise); Class II: describes patients with heart disease that causes clinical signs only during strenuous exercise; Class III: describes patients with heart disease that causes clinical signs with routine daily activities or mild exercise; and Class IV: describes patients with heart disease that causes severe clinical signs even at rest.

In additional embodiments, provided here in are methods of distinguishing non-diseased subjects from diseased subjects with the mitral valve disease or condition wherein the non-diseased subjects correspond to stage A subjects as classified by the ACVIM classification system and the diseased subjects with the mitral valve disease or condition correspond to stages B1, B2, C and D subjects as classified by the ACVIM classification system.

The ACVIM system describes four basic stages of heart disease and failure: Stage A: patients at high risk for developing heart disease but that currently have no identifiable structural disorder of the heart; Stage B: patients with structural heart disease (e.g., the typical murmur of mitral valve regurgitation is present), but that have never developed clinical signs caused by heart failure (because of important clinical implications for prognosis and treatment, the panel further subdivided Stage B into Stage B1 and B2). Stage B1: asymptomatic patients that have no radiographic or echocardiographic evidence of cardiac remodeling in response to CVHD. Stage B2: asymptomatic patients that have hemodynamically significant valve regurgitation, as evidenced by radiographic or echocardiographic findings of left sided heart enlargement. Stage C: patients with past or current clinical signs of heart failure associated with structural heart disease. Stage D: patients with end-stage disease with clinical signs of heart failure caused by CVHD that are refractory to standard therapy.

The pathology of the heart begins with ISACHC Class I, NYHA Class I and ACVIM stage B2 in which cardiac murmur or cardiac chamber enlargement, but no clinical symptoms are present (ISACHC Class I or asymptomatic/occult/preclinical stage). Clinical symptoms become manifest in the course of progression of the disease (ISACHC Class II or III, NYHA class II, III or IV, ACVIM stage C and D).

Provided herein are methods of selecting a panel for use in disease diagnosis comprising the steps of: selecting a group of miRNA molecules the differential expression of which may be associated with a disease condition; applying one or more predictive classification models to be able to predict the disease condition; and using the one or more predictive classification models to reduce the number of miRNAs in the panel to a minimum number to provide a panel of miRNAs that still produces a result. Also provided herein are methods of selecting a nucleic acid panel for use in differential diagnosis between a healthy and MMVD subjects.

In another embodiment, the invention provides methods for distinguishing non-diseased subjects from diseased subjects with mitral valve (MR) and MMVD diseases. MR disease can take years to develop for first diagnosis, and can sometimes present with congestive heart failure.

### C. Cardiomyopathy

Cardiomyopathies are defined as diseases of the myocardium associated with cardiac dysfunction (Richardson P, et al. Circulation 1996; 93:841-842). The new classification of cardiomyopathies by the World Health Organization is by dominant pathophysiology or, where possible, by aetiological/pathogenetic factors.

i. Dilated Cardiomyopathy (DCM)

Dilated cardiomyopathy is defined as being characterized by dilatation and impaired contraction of the left ventricle or both ventricles1. The diagnosis of idiopathic dilated cardiomyopathy (DCM) requires the active exclusion of other cardiac, pulmonary or systemic disease which may secondarily induce a similar phenotype (Cobb M A. J Small Anim Pract., 1992; 33:113-118).

The invention provides methods of assessing and diagnosing DCM. The invention also provides methods for differentially diagnosing MMVD from DCM. Clinical signs of MMVD are similar to DCM. DCM includes left apical systolic heart murmur, left-sided congestive heart failure (including tachypnea, cough, exercise intolerance, syncope, and respiratory distress), arrhythmias, and poor appetite. Therefore,

DCM is a primary disease of cardiac muscle that results in mechanical dysfunction and/or electrical dysfunction and a decreased ability of the heart to generate pressure to pump blood through the vascular system.

Traditionally, dilated cardiomyopathy (DCM) is diagnosed when clinical signs become apparent as caused by cardiac dysfunction. These include signs such as panting, loss of energy, persistent cough, exercise intolerance and heart murmur, combined with a demonstration of structural and functional changes to the heart using imaging that indicates an enlarged heart with hypertrophic changes to the heart muscle's structure, and dysfunction, preferably using an ultrasound scan by a veterinary cardiologist. Radiographs can also be used to demonstrate an enlarged heart, but this will not provide evidence of dysfunction and is therefore not considered definitive.

Among causes, genetics plays a role with certain large breeds of dogs most predisposed to developing the condition as they age. However, taurine deficiency has also been demonstrated to cause DCM. Since 2018, certain diets have been associated with an increase in the prevalence of the condition in dogs across breed and age. These diets are typically so-called grain-free, legume-rich diets, especially those containing high inclusion levels of peas and/or lentils. Taurine deficiency does not appear to be the cause in most cases. More than 1000 cases have been reported to the FDA. The challenge is that by the time dogs show clinical signs, the heart's structural changes may have reached the point of heart failure, thus resulting in a worsening prognosis. There is a need, therefore, for methods that can help with early detection or diagnosis of DCM. There, further, is a need for providing treatment and/or customized recommendations to treat or t reduce the health risks associated with DCM.

DCM may be defined by: (i) left ventricular dilation; (ii) reduced systolic function; and (iii) increased sphericity of the left ventricle. Development of DCM is slow and few clinical signs manifest over time. As DCM progresses, signs include lethargy, anorexia, shallow breathing, sudden fainting, and potential death. The clinical stage of DCM may be subtle and is typically characterized by signs of congestive heart failure, with or without cardiac arrhythmias. DCM occurs most commonly in medium and large breed dogs and causes progressive congestive heart failure. The functional classification for heart failure is summarized by the New York Heart Association (NYHA) classification, summarized in Table 1.

TABLE 1
Functional classification for heart failure

| | |
|---|---|
| Class I | Patients with asymptomatic heart disease (e.g., CVHD is |
| | present but no clinical signs are evident, even with exercise). |
| Class II | Patients with heart disease that causes clinical signs only |
| | during strenuous exercise. |
| Class III | Patients with heart disease that causes clinical signs with |
| | routine daily activities or mild exercise. |
| Class IV | Patients with heart disease that causes sever clinical signs, |
| | even at rest. |

The ACVIM developed a newer classification system to more objectively categorize animals in the course of their heart disease, linking the severity of the signs to appropriate treatments at each stage. According to the ACVIM, the stages include:
Stage A: Animals at high risk for heart disease (no disease present).
Stage B: A murmur is heard but there are no visible signs of heart failure.
   Stage B1: The heart does not appear enlarged or changed on X-ray.
   Stage B2: The heard appears enlarged or changed on X-ray.
Stage C: Evidence of heart failure is visible and treatment is necessary.
Stage D: Heart failure is getting hard to manage and is not responding to standard treatment

Dilated cardiomyopathy has been reported to have an overall prevalence of 0.5%, based on the Veterinary Medical Database at Purdue University for the period 1986-1991 (Sisson D D, Thomas W P. Myocardial diseases. In Ettinger S J, Feldman E C ed. Textbook of Veterinary Internal Medicine, 4th edition. Philadelphia, WB Saunders; 1995:995-1032). This only included dogs with clinical signs referable to heart disease, particularly congestive heart failure. Further analysis showed higher prevalence in pedigree dogs (0.65%) compared with cross-breeds (0.16%), and prevalence in certain specific breeds was very high (e.g. Deerhounds 6.0%; Dobermanns, 5.8%, Irish Wolfhounds, 5.6%; great Danes, 3.9%; Boxers, 3.4%; Newfoundlands, 1.3%). Large and giant breed dogs are most predisposed to develop DCM, with the exception of spaniel breeds (especially Cocker Spaniels (English and American)). Prevalence increases with age, and males are usually reported as being overrepresented (Sisson D D, Thomas W P. Myocardial diseases. In Ettinger S J, Feldman E C ed. Textbook of Veterinary Internal Medicine, 4th edition. Philadelphia, WB Saunders; 1995:995-1032) in the population of dogs with congestive heart failure. Prevalence rates are much higher in prospective studies screening for the presence of DCM, when dogs without clinical signs may be detected (e.g. Dobermanns, 63.2% (O'Grady M R, Horne R. Proceedings of the 16th ACVIM Forum 1998:689) Irish Wolfhounds, 24.2% (Vollmar A C. J Am Anim Hosp Assoc 2000; 36:125-132); Newfoundlands, 17.6% (Dukes-McEwan J. PhD thesis. University of Edinburgh. 1999). It should, however, be noted that concentration on certain family lines in these studies may over-estimate actual population prevalence. In these prospective studies, there is not usually a marked sex-predisposition reported, although male dogs may show an earlier onset of congestive heart failure (Calvert C A. In Bonagura J D ed. Kirk's Current Veterinary Therapy. Small Animal Practice XII. Philadelphia, WB Saunders; 1995:799-806).

Provided herein are methods of selecting a panel for use in disease diagnosis comprising the steps of: selecting a group of miRNA molecules the differential expression of which may be associated with a disease condition; applying one or more predictive classification models to be able to predict the disease condition; and using the one or more predictive classification algorithms to reduce the number of miRNAs in the panel to a minimum number to provide a panel of miRNAs that still produces a result. Also provided herein are methods of selecting a nucleic acid panel for use in differential diagnosis between a healthy and DCM subjects. Additionally, methods of selecting a nucleic acid panel for use in differential diagnosis between MMVD and DCM subjects are provided herein.

ii. Hypertrophic Cardiomyopathy (HCM)

Hypertrophic Cardiomyopathy (HCM) is the most common heart condition in cats. It is characterized by thickening of cardiac muscle, preventing the heart from filling properly and decreasing its efficiency. This can lead to various symptoms, including arrhythmia, heart murmur, lethargy, breathing difficulty, blood clots, difficulty walking and death. Due to the profound cardiac remodeling that occurs during HCM, it is possible that the miRNA panel designed to detect cardiac remodeling in canine myxomatous mitral valve disease (MMVD) may also be suitable for the diagnosis of HCM despite being for a different species.

### E. Identification of Target Sequences

The present invention relates to a method for detecting the presence or amount of a target polynucleotide (nucleic acid sequence) from the host's response to MMVD in a sample. The target polynucleotide is a virulence determinant. In a preferred embodiment, the target polynucleotide is a miRNA. The invention is also directed to a method of detecting the presence of a disease or infection state in a mammal, by detecting the presence or amount of a target miRNA, wherein the presence or amount of the target miRNA identifies the disease state. Thus, the invention relates to diagnostic compositions and methods for detecting MMVD. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

In another embodiment, the invention provides a method for capturing, detecting and quantifying miRNA from its reverse transcribed cDNA. miRNA is extracted from the provided biological sample using commercially available miRNA specific extraction kits and the manufacturer's recommended protocol (e.g. Qiagen miRNeasy Serum/Plasma Kit). From the extracted miRNA, cDNA is reverse transcribed and amplified using commercially available miRNA to cDNA specific extraction kits and the manufacturer's recommended protocol (e.g. TaqMan Advanced miRNA cDNA Synthesis Kit). The resulting reverse transcribed cDNA of the miRNA may be captured and/or detected using the universal sequences added at both the 5′ and 3′ ends and the cDNA product may undergo universal pre-amplification and/or amplification using a single pair of universal forward and reverse primers. The relative expression levels of specific miRNA, which form part of the defined diagnostic panel, are inferred through the relative expression levels of their respective cDNA, i.e. detection by proxy. This can be performed via numerous traditional DNA detection methods, such as qPCR or Next Generation sequencing, or via newer multiplexing techniques such as beads capture technologies such as the Luminex xMAP system.

The invention described herein utilizes large-scale identification of disrupted genes and the use of bioinformatics and AI to select mutants that could be characterized in animals.

F. Multiplex miRNA Profiling

The present invention uses multiplex miRNA profiling without RNA purification. Accuracy of miRNA profiling is enhanced when sample processing can be kept to a minimum, avoiding steps such as RNA purification that can introduce bias and inaccuracies. The present invention used a multiplex circulating miRNA assay that enables the profiling of a plurality of miRNAs in the same well directly from the sample, with no need for RNA purification. In one embodiment, the assay uses FirePlex® particles, which enable the multiplex capture of miRNAs with picomolar sensitivity and high specificity. The FirePlex® particles contain three distinct functional regions that are separated from each other by inert spacer regions. The central region of each particle is known as a central analyte or miRNA quantification region which contains miRNA probes that can capture target miRNAs. The central region of the particle comprises a reporter dye. The two end regions of each particle act as two halves of a barcode that distinguish between different particles. Detection is carried out using a flow cytometer to detect miRNA molecules that emit fluorescence that is proportional to their abundance in the sample. The flow cytometer was used to detect the fluorescence signal from the center of each particle through the reporter dye. Each miRNA that was used was given a unique code (up to 70 different codes were possible). The data that was obtained from the mixture of particles could then be attributed to the miRNAs by identification of the code.

The disease is selected from the group consisting of MMVD and related conditions.

The sample or blood sample refers to tissue or organ sample, blood, cell-free blood such as serum and plasma, urine, saliva, milk and cerebrospinal fluid sample.

From the results of the experiments below, a differentiation in expression levels of miRNA was identified when comparing healthy animals with animals that have MMVD.

### G. Predictive Modelling

Provided herein are methods using predictive modelling to investigate the use of miRNA profiles to predict the presence or absence of disease. A group of healthy and unhealthy animals were obtained and tested to determine the level of miRNA expression in samples from these animals. The data obtained was then used to train the models.

Disclosed herein are predictive classifier models, computer implemented systems, machine learning systems and methods thereof for classifying asymptomatic patients into a risk category for having or developing cardiac disease and/or classifying a patient with an increased risk of having or developing a specific cardiac disease and differentially diagnosing stages of the cardiac disease. Also disclosed are methods of using the predictive classifying models to differentially diagnose specific cardiac diseases.

Fifteen machine learning models were fitted and compared with the aim of obtaining the best predictions of the disease outcome. Formal assessment of performance was conducted by computing a number of performance statistics based on 5-time repeated 10-fold cross-validation. Cross-validation was useful to obtain more realistic model performance measures from the training data.

Data from the FirePlex analysis from each of the miRNA molecules from Table 2 was fitted to each of the models.

In some embodiments, the machine learning system generates a predictive classifier model that may be static. In other words, the predictive classifier model is trained and then its use is implemented with a computer implemented system wherein patient data (e.g. biomarker marker measurements) are input and the predictive classifier model provides an output that is used to classify patients.

In other embodiments, the predictive classifier model is further trained and improved by the machine learning system comprising (1) obtaining one or more test results from the diagnostic testing which confirm or deny the presence and/or stage of cardiac disease in the patient, (2) incorporating the one or more test results into the training data for further training of the predictive classifier model of the machine learning system; and (3) generating an improved predictive classifier model by the machine learning system.

In additional embodiments provided herein a predictive classifier model is provided to predict an increased risk of having or developing cardiac disease for an asymptomatic patient, staging the cardiac disease for diseased patients, and differentially diagnosing the cardiac disease to predict the disease outcome. In embodiments, this classifier model is generated by one or more machine learning system using training data that comprises values of a panel of one or more biomarkers, and diagnostic indicator for a population of patients.

In one embodiment, the predictive classifier model classifies the patient in an increased risk category using the measured values of a panel of biomarkers. In other embodiments, the predictive classifier model classifies the patient's disease stage category using the measured values of a panel of biomarkers. In exemplary embodiments, the output is a probability value, wherein the threshold is set to separate patients into a disease risk category for one cardiac disease from an disease risk category of another cardiac disease. In one example the predictive classifier model assesses and differentially diagnoses patients with MMVD from patients with MR. In another example, the predictive classifier model differentially diagnoses patients with DCM from patients with HCM.

Disclosed herein is a machine learning system comprising at least one processor for predicting an increased risk for cardiac disease. In certain embodiments, the processor is configured to obtain measured values of a panel of biomarkers in a sample from a patient, wherein a value of a biomarker corresponds to a level of the biomarker in the sample, and generates a predictive classification model by the machine learning system to assess and differentially diagnose cardiac disease, wherein the predictive classifier model is generated by the machine learning system using training data that comprises values from a panel of at least one or more biomarkers.

Embodiments of the present invention further provide for an apparatus for assessing and differentially diagnosing a cardiac disease. The apparatus may comprise a processor configured to execute computer readable media instructions (e.g., a computer program or software application, e.g., a machine learning system), to receive the concentration values from the evaluation of biomarkers in a sample and, in combination with other risk factors (e.g., medical history of the patient, including publicly available sources of information pertaining to a risk of developing cardiac disease, etc.) a risk score may be generated and compared to a grouping of a stratified cohort population comprising multiple categories.

The apparatus can take any of a variety of forms, for example, a handheld device, a tablet, or any other type of computer or electronic device. The apparatus may also comprise a processor configured to execute instructions e.g., a computer software product, an application for a handheld device, a handheld device configured to perform the method, a world-wide-web (WWW) page or other cloud or network accessible location, or any computing device. In other embodiments, the apparatus may include a handheld device, a tablet, or any other type of computer or electronic device for accessing a machine learning system provided as a software as a service (SaaS) deployment. Accordingly, the correlation may be displayed as a graphical representation, which, in some embodiments, is stored in a database or memory, such as a random access memory, read-only memory, disk, virtual memory, and the like. Other suitable representations or exemplifications known in the art may also be used.

The apparatus may further comprise a storage means for storing the correlations, an input means, and a display means for displaying the status of the subject in terms of the particular medical condition assessed. The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tube (CRT), a digital screen, a light-emitting diode (LED), a liquid crystal display (LCD), an X-ray, a compressed digitized image, a video image, or a hand-held device. The apparatus can further comprise or communicate with a database, wherein the database stores the correlation of factors and is accessible to the user.

In another embodiment of the present invention, the apparatus is a computing device, for example, in the form of a computer or hand-held device that includes a processing unit, memory, and storage. The computing device can include or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, stylus or the like. The output can also be one or several devices, such as a video display, a printer, an audio output device, a touch stimulation output device, a screen reading output device or the like. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

Artificial intelligence systems include computer systems configured to perform tasks usually accomplished by humans, e.g., speech recognition, decision making, language translation, image processing and recognition, and the like. In general, artificial intelligence systems have the capacity to learn, to maintain and to access a large repository of information and to perform reasoning and analysis in order to make decisions. Such systems also have the ability to self-correct.

Artificial intelligence systems may include knowledge representation systems and machine learning systems. Knowledge representation systems generally provide structure to capture and encode information used to support decision making. Machine learning systems are capable of analyzing data to identify new trends and patterns in the data. For example, machine learning systems may include neural networks, induction algorithms, genetic algorithms, and the like and may derive solutions by analyzing patterns in data.

In certain embodiments, the present classifier models comprise an algorithm such as a support vector machine, a decision tree, a random forest, a neural network, a deep learning neural network, a logistic regression or a pattern recognition algorithm. The present classifier models may be used to classify an individual patient into one of a plurality of categories, e.g., a category indicative of a likelihood of a cardiac disease, a category indicating the stage of the cardiac disease, or a category differentiating between cardiac diseases. Inputs to the classifier model may include a panel of biomarkers associated with the presence of a cardiac disease as well as clinical parameters.

A variety of machine learning models are available, including support vector machines, decision trees, random forests, neural networks or deep learning neural networks. Generally, support vector machines (SVMs) are supervised learning models that analyze data for classification and regression analysis. SVMs may plot a collection of data points in n-dimensional space (e.g., where n is the number of biomarkers and/or clinical parameters), and classification is performed by finding a hyperplane that can separate the collection of data points into classes. In some embodiments, hyperplanes are linear, while in other embodiments hyperplanes are non-linear. SVMs are effective in high dimensional spaces; are effective in cases in which the number of dimensions is higher than the number of data points; and generally work well on data sets with clear margins of separation.

Decision trees are a type of supervised learning algorithm also used in classification problems. Decision trees may be used to identify the most significant variable that provides the best homogenous set of data. Decision trees split groups of data points into one or more subsets, and then may split each subset into one or more additional categories, and so forth until forming terminal nodes (e.g., nodes that do not split). Various algorithms may be used to decide where a split occurs, including a Gini Index (a type of binary split), Chi-Square, Information Gain, or Reduction in Variance. Decision trees have the capability to rapidly identify the most significant variables among a large number of variables, as well as identify relationships between two or more variables. Additionally, decision trees can handle both numerical and non-numerical data. This technique is generally considered to be a non-parametric approach, e.g., the data does not have to fit a normal distribution.

Random forest (or random decision forest) is a suitable approach for both classification and regression. In some embodiments, the random forest method constructs a collection of decision trees with controlled variance. Generally, for M input variables, a number of variables (nvar) less than M is used to split groups of data points. The best split is selected and the process is repeated until reaching a terminal node. Random forest is particularly suited to process a large number of input variables (e.g., thousands) to identify the most significant variables. Random forest is also effective for estimating missing data.

Neural nets (also referred to as artificial neural nets (ANNs)) are described throughout this application. A neural net, which is a non-deterministic machine learning technique, utilizes one or more layers of hidden nodes to compute outputs. Inputs are selected and weights are assigned to each input. Training data is used to train the neural networks, and the inputs and weights are adjusted until reaching specified metrics, e.g., a suitable specificity and sensitivity.

ANNs may be used to classify data in cases in which correlation between dependent and independent variables is not linear or in which classification cannot be easily performed using an equation. More than 25 different types of ANNs exist, with each ANN yielding different results based on different training algorithms, activation/transfer functions, number of hidden layers, etc. In some embodiments, more than 15 types of transfer functions are available for use with the neural network. Prediction of the likelihood of having cardiac disease is based upon one or more of the type of ANN, the activation/transfer function, the number of hidden layers, the number of neurons/nodes, and other customizable parameters.

Deep learning neural networks, another machine learning technique, are similar to regular neural nets but are more complex (e.g., typically have multiple hidden layers) and are capable of automatically performing operations (e.g., feature extraction) in an automated manner, generally requiring less interaction with a user than a traditional neural net.

In some embodiments, inputs may be selected in order to improve the performance of the classifier model. For example, rather than picking the set of inputs that achieves the highest possible sensitivity with a clinically relevant specificity such as such as 80% or greater, the inputs are selected to reach a sensitivity threshold (e.g., 80% or greater), and once reaching this threshold, the inputs are selected to optimize performance of the classifier model, thereby improving the performance of the classifier model.

Accordingly, systems, methods and computer readable media are presented herein regarding using a machine learning system, e.g. to generate a classifier model, to identify a patient's risk of having cardiac disease. A set of data comprising a plurality of patient records, each patient record including a plurality of parameters and corresponding values for a patient, and wherein the set of data also includes a diagnostic indicator indicating whether or not the patient has been diagnosed with a cardiac disease is stored in a memory, accessible by the classifier model or machine learning system. The plurality of parameters includes various biomarkers, clinical factors and other factors which may be selected as inputs into the classifier model. The diagnostic indicator is an affirmative indicator that the patient has a particular cardiac disease or the stage of the cardiac disease. A subset of the plurality of parameters is selected for inputs into the machine learning system, wherein the subset includes a panel of at least two different biomarkers and at least one clinical parameter, such as weight.

In order to train the classifier model generated by the machine learning system, the set of data (e.g. longitudinal) is randomly partitioned into training data and validation data. The classifier model is generated using the machine learning system based on the training data, the subset of inputs and other parameters associated with the machine learning system as described herein. It is determined whether the classifier meets certain performance criteria, such as a predetermined Receiver Operator Characteristic (ROC) statistic, specifying a sensitivity and a specificity, for correct classification of patients.

A user, with access to a computing device with the static classifier model, may enter input values corresponding to a patient into the computing device. The patient may then be classified, using the static classifier, into a category indicative of a cardiac disease or into another category indicative of the stage of the cardiac disease. The system may then send a notification to the user (e.g., a veterinarian) recommending additional diagnostic testing when the subject is classified into the category indicative of a cardiac disease or a stage of a cardiac disease.

In some embodiments, the classifier model generated by the machine learning system may be continuously trained over time. Test results obtained from the diagnostic testing, which confirm or deny the presence of cardiac disease, may be incorporated into the training data set for further training of the machine learning system, and to generate an improved classifier by the machine learning system.

In some embodiments, although the machine learning system can evolve over time to make more accurate predictions, the machine learning system may have the capability to deploy improved predictions on a scheduled basis. In other words, the techniques used by the machine learning system to determine risk may remain static for a period of time, allowing consistency with regard to determination of a risk score. At a specified time, the machine learning system may deploy updated techniques that incorporate analysis of new data to produce an improved risk score. Thus, the machine learning systems described herein may operate: (1) in a static manner; (2) in a semi-static manner, in which the classifier is updated according to a prescribed schedule (e.g., at a specific time); or (3) in a continuous manner, being updated as new data is available.

### H. Measuring Biomarkers in a Sample

As part of the present method, a panel of markers from a subject may be measured. There are many methods known in the art for measuring either gene expression (e.g., mRNA) or the resulting gene products (e.g., polypeptides or proteins) that can be used in the present methods, and known to one of skill in the art.

In one embodiment, gene expression of markers (e.g., mRNA) is measured in a sample from a subject. For example, gene expression profiling methods for use with paraffin-embedded tissue include quantitative reverse transcriptase polymerase chain reaction (qRT-PCR), however, other technology platforms including mass spectroscopy and DNA microarrays can also be used. These methods include, but are not limited to, PCR, Microarrays, Serial Analysis of Gene Expression (SAGE), and Gene Expression Analysis by Massively Parallel Signature Sequencing (MPSS).

Any methodology that provides for the measurement of a marker or panel of markers from a subject is contemplated for use with the present methods. In certain embodiments, the sample from the subject is a bodily fluid such as blood, serum, plasma or a part or fraction thereof. In other embodiments, the sample is a blood or serum and the markers are proteins measured therefrom. In yet another embodiment, the sample is a tissue section and the markers are mRNA expressed therein. Many other combinations of sample forms from the subjects and the form of the markers are contemplated.

A panel can comprise any number of biomarkers as a design choice, seeking, for example, to maximize specificity or sensitivity of the classifier model. Hence, the present methods may ask for presence of at least one of two or more biomarkers, three or more biomarkers, four or more biomarkers, five or more biomarkers, six or more biomarkers, seven or more biomarkers, eight biomarkers or more as a design choice.

Thus, in one embodiment, the panel of biomarkers may comprise at least two, at least three, at least four, at least five, at least six, at least seven, at least eight, at least nine or at least ten or more different markers. In one embodiment, the panel of biomarkers comprises about two to ten different markers. In another embodiment, the panel of biomarkers comprises about four to eight different markers. In yet another embodiment, the panel of markers comprises about six or about seven different markers.

Generally, a sample is committed to the assay and the results can be a range of numbers reflecting the presence and level (e.g., concentration, amount, activity, etc.) of presence of each of the biomarkers of the panel in the sample.

The choice of the markers may be based on the understanding that each marker, when measured and normalized, contributed equally as an input variable for the classifier model. Thus, in certain embodiments, each marker in the panel is measured and normalized wherein none of the markers are given any specific weight. In this instance each marker has a weight of 1.

### H. Kits

Also provided herein is a kit for use in performing the method of the first aspect comprising means for determining the level of expression of each one of the following miRNA molecules: cfa-miR-30d-5p, cfa-miR-128-3p, cfa-miR-133a-3p, cfa-miR-133b-3p, cfa-miR-142-5p, cfa-miR-206-3p, cfa-miR-320-3p, cfa-miR-423a-5p, cfa-miR-499-5p, cfa-let-7b-5p, cfa-let-7e-5p, cfa-let-7i-5p, cfa-miR-29a-3p and cfa-miR-486-5p.

There is also provided a method of selecting a panel for use in disease diagnosis comprising the steps of: (a) selecting a group of miRNA molecules the differential expression of which may be associated with a disease condition; (b) applying one or more predictive classification models to be able to predict the disease condition; and (c) using the one or more predictive classification models to reduce the number of miRNAs in the panel to a minimum number to provide a panel of miRNAs that still produces a result.

### EXAMPLES

### Methods and Materials

This study included surplus blood samples from cases assessed by the cardiology clinic at Centre 1 (Cardiology Service of the Small Animal Teaching Hospital, University of Liverpool, Liverpool, UK), and surplus blood samples submitted to the diagnostic laboratory at Centre 2 (SRUC Veterinary Services, SRUC, Edinburgh, UK) during the study period. Blood samples were taken as clinically indicated testing. No blood was taken specifically for this study. Ethical approval was granted for the study at both sites, and consent obtained for research use of samples.

Sample collection and exclusion criteria: A total of 123 canine blood samples (serum or plasma) were collected from unique cases between September 2020 and December 2021. Additional sample data included various animal characteristics, including breed, age, sex, and neuter status for all samples. All MMVD cases (n=73) were sourced from Centre 1 and confirmed by echocardiography by a specialist veterinary cardiologist or a cardiology resident under supervision of a Diplomate. Data from the physical examination, auscultation findings were retrieved by a retrospective review of medical records. Medication received at the time of presentation was recorded. Cases were categorized into pre-clinical (Stage B1/B2) and clinical (Stage C/D) using ACVIM guideline definitions (Keene, B W, et al. J Vet Intern Med. 33:1127 1140 (2019)). The concentrations of currently applied cardiac blood biomarker levels (NT-proBNP and cTnI) were also available for a number of cases. The 2nd generation NT-proBNP assay was used (IDEXX, Wetherby). Concentration of >900 pmol/L was defined as abnormal, based on the laboratory reference ranges. Troponin I was either assayed by Immulite 2000 (Siemens) with abnormal values defined as >0.15 ng/ml or the Beckman Coulter Access high sensitivity assay (IDEXX, Wetherby), with abnormal values defined a >0.07 ng/ml, based on the respective laboratory reference ranges. Results were dichotomized to normal or abnormal values (i.e. exceeding the respective reference range) in both control and MMVD cases. Values above the reference ranges were considered to reflect cardiac disease, specifically MMVD, since other cardiac disease and systemic conditions had been excluded. Any MMVD cases that had history or clinical signs of concurrent disease were excluded from the study.

Control cases from the Centre I were subject to the same examinations for selection (n=18), and those from Centre 2 selected on the basis having no known current pathology and normal biochemistry and haematology parameters to serve as further controls (n=32). Blood samples had been taken for various tests as clinically indicated, and surplus blood only was submitted as part of this study. The results of clinical pathology testing were reviewed to assess general health, and to identify any significant comorbidity and exclusion criteria.

Clinical examination, auscultation and echocardiography of MMVD cases: Physical examination findings were recorded at the time of examination, and the following data were retrieved: heart rate and rhythm, respiratory rate and cardiac and pulmonary auscultation findings. The presence, point of maximal intensity and grade of any heart murmur was recorded, noting radiation. Systolic blood pressure was measured routinely in all MMVD cases by Doppler sphygmomanometryas recommended (Acierno M J, et al., J Vet Intern Med. 32 (6): 1803-1822 (2018)). Echocardiography was carried out on each dog included in the MMVD group within an hour after auscultation by the same clinician who performed the auscultation, with a GE Vivid 7 or Vivid E95 machine, with a 3- to 8-MHz phased-array transducer. A complete Doppler echocardiographic assessment was carried out, but data retrieved to allocate the ACVIM stage (Keene, B W, et al., J Vet Intern Med. 33:1127 1140 (2019)) for this study included the right parasternal 2D short axis left atrium to aortic (LA/Ao) ratio, with an optimised left atrial diameter, in early diastole, including aortic valves, the first frame after they closed (in early diastole) (Hansson K, et al., Vet Radiol Ultrasound. 43 (6): 568-75 (2002)). Left ventricular M-modes were obtained, with M-mode cursor positioned on right parasternal short axis views at the level of tips of papillary muscle, to bisect the left ventricular cavity. The left ventricular internal diameter in diastole was normalised for body weight by allometric scaling (Cornell C C, et al., J Vet Intern Med. 18 (3): 311-21 (2004)).

From right parasternal long axis 4 and 5 chamber views, optimising the left atrium and the aorta respectively, the maximal left atrial diameter (LAmax) at the end of ventricular systole and the diameter of the aortic annulus between open aortic valve leaflets were measured, and the LAmax/aortic annulus ratio calculated (Strohm L E, et al., J Vet Cardiol. 20 (5): 330-342 (2018)). From right parasternal long axis views optimising the left ventricular length and area, Simpson's method of discs (SMOD) was used to determine the end-diastolic and end-systolic left ventricular volumes (EDV and ESV respectively; mLs). The ejection fraction percentage was calculated as ((EDV−ESV)/EDV)×100. The EDV and ESV were normalised for body weight by dividing by weight in kg (Wess G, et al., J Vet Intern Med. 35 (2): 724-738 (2021)). From the left apical 4 chamber view, aligned for transmitral flow, spectral Doppler was obtained and the mitral E wave velocity was measured. From the apical 5 chamber view, with the sample volume between transmitral flow and left ventricular outflow tract (LVOT flow, spectral Doppler was obtained, and the isovolumic relaxation time (IVRT) measured at end of LVOT flow and start of the mitral E wave. The E/IVRT was measured as an estimate of left sided filling pressures (Schober K E, et al., J Vet Intern Med. 24 (6): 1358-68 (2010)). If thoracic radiographs had been obtained, the vertebral heart sum was measured from the right lateral view (Buchanan J W, Bücheler J,. J Am Vet Med Assoc. 15; 206 (2): 194-9 (1995)). Presence of cardiogenic pulmonary oedema was noted.

Clinical examination, auscultation, blood pressure assessment and echocardiography of all MMVD cases for the study was performed by one or more of the authors. The medication dogs were on at the time of the assessment was noted, but medication subsequently prescribed as a result of the assessment and sampling is not included.

MicroRNA expression profiling: MicroRNAs to be included in the heart disease-specific miRNA panel were selected through a review of manuscripts identified in a PubMed-based literature search which included heart disease and mitral valve disease research in humans, dogs, cats and rodents. This was supplemented and adjusted using mirPath v3 database (Vlachos I S, et al., 1; 43 (W1): W460-6 (2015)) to predict likely roles of microRNAs based on the pathology of mitral valve disease and activated disease pathways. This resulted in a sequence of 20 miRNAs noted to have altered expression during heart disease which were mapped to MIRBase for confirmation of sequences and nomenclature (Griffiths-Jones S. miRBase: The MicroRNA Sequence Database. In: MicroRNA Protocols [Internet]. New Jersey: Humana Press; 2006 [cited 2023 May 5]. p. 129-38) (Table 2). The most stable expressed miRNAs in an exploratory data set of canine and feline samples (n=556) were selected as normalizer miRNAs using the geNorm function of Fireplex Analysis Workbench 2.0.274 (Abcam, Cambridge, UK) (Vandesompele J, et al., 2002 June; 3 (7): 1-2 (2002)) (cfa-mir-17-5p, cfa-mir-130b-3p, cfa-mir-20a-5p, cfa-mir-23a-3p, cfa-mir-26a-5p). These included miRNAs previously suggested to be involved in canine cardiac disease but were found to have low variance in our dataset. Lastly, three off-species miRNAs were also included to act as background controls (Table 2). These sequences were used to design a custom 23-plex panel for the Fireplex miRNA platform (Abcam, Cambridge, UK). For expression profiling, 50 μL aliquots of sera from each sample were incubated with Fireplex capture microbead particles specific to the miRNA targets and processed following the manufacturer's instructions with optimised hybridization, melt-off and capture temperatures of 39° C., 62° C. and 39° C., respectively. The mean florescence intensities (MFI) of miRNA-specific particles per sample was measured to quantify miRNA expression using a Novocyte flow cytometer and Novosampler Pro software (Agilent, Santa Clara, USA). Raw FCS files were exported to Fireplex Analysis Workbench 2.0.274 (Abcam, Cambridge, UK) and normalized expression values prepared using the ‘geomean’ function with the pre-selected normalisers identified in the preliminary dataset.

TABLE 2
Summary information for the profiling panel indicating mature
sequence and available predicted signaling pathways targeted by each miRNA (p < 0.05).

| | SEQ | | | | |
|---|---|---|---|---|---|
| | ID | | Signalling | | |
| miRNA | NO: | Mature Sequence | Pathways | References | |
| cfa-miR-30b- | 1 | UGUAAACAUCCUACA | p53, Wnt | Ghilardi, Sara, et al. | Bagardi, Mara, et al. |
| 5p | | CUCAGCU | | “Prospective pilot | “Circulating miR-30b-5p |
| | | | | study on the | is upregulated in |
| | | | | predictive | Cavalier King Charles |
| | | | | significance of | Spaniels affected by |
| | | | | plasma miR-30b-5p | early myxomatous |
| | | | | through the study of | mitral valve |
| | | | | echocardiographic | disease.” bioRxiv (2022): |
| | | | | modifications in | 2022-03. |
| | | | | Cavalier King Charles | |
| | | | | Spaniels affected by | |
| | | | | different stages of | |
| | | | | myxomatous mitral | |
| | | | | valve disease: The | |
| | | | | PRIME study.” Plos | |
| | | | | one 17.12 (2022): | |
| | | | | e0274724. | |
| cfa-miR-30d- | 2 | UGUAAACAUCCCCGAC | Hippo, p53, | Yang, Vicky K., et al. | |
| 5p | | UGGAAGCU | Wnt | “Dysregulation of | |
| | | | | valvular interstitial | |
| | | | | cell let-7c, miR-17, | |
| | | | | miR-20a, and miR- | |
| | | | | 30d in naturally | |
| | | | | occurring canine | |
| | | | | myxomatous mitral | |
| | | | | valve disease.” PloS | |
| | | | | one 13.1 (2018): | |
| | | | | e0188617. | |
| cfa-miR-128- | 3 | UCACAGUGAACCGGU | | Jung, SeungWoo, | Bagardi, Mara, et al. |
| 3p | | CUCUUU | | and Amy Bohan. | “Circulating miR-30b-5p |
| | | | | “Genome-wide | is upregulated in |
| | | | | sequencing and | Cavalier King Charles |
| | | | | quantification of | Spaniels affected by |
| | | | | circulating | early myxomatous |
| | | | | microRNAs for dogs | mitral valve disease.” |
| | | | | with congestive | bioRxiv (2022): 2022-03. |
| | | | | heart failure | |
| | | | | secondary to | |
| | | | | myxomatous mitral | |
| | | | | valve degeneration.” | |
| | | | | American journal of | |
| | | | | veterinary research | |
| | | | | 79.2 (2018): 163- | |
| | | | | 169. | |
| cfa-miR- | 4 | UUGGUCCCCUUCAAC | | Shan, Hongli, et al. | Li, Hongli, et al. |
| 133a-3p | | CAGCUGU | | “Downregulation of | “Expression of miR-133 |
| | | | | miR-133 and miR- | and miR-30 in chronic |
| | | | | 590 contributes to | atrial fibrillation in |
| | | | | nicotine-induced | canines.” Molecular |
| | | | | atrial remodelling in | medicine reports 5.6 |
| | | | | canines.” | (2012): 1457-1460. |
| | | | | Cardiovascular | |
| | | | | research 83.3 (2009): | |
| | | | | 465-472. | |
| cfa-miR- | 5 | UUUGGUCCCCUUCAA | | Hulanicka, | |
| 133b-3p | | CCAGCUA | | Magdalena, et al. | |
| | | | | “Plasma miRNAs as | |
| | | | | potential biomarkers | |
| | | | | of chronic | |
| | | | | degenerative | |
| | | | | valvular disease in | |
| | | | | Dachshunds.” BMC | |
| | | | | veterinary research | |
| | | | | 10.1 (2014): 1-8 | |
| cfa-miR-142- | 6 | CCCAUAAAGUAGAAAG | p53 | Jung S, Bohan A. | |
| 5p | | CACUA | | Genome-wide | |
| | | | | sequencing and | |
| | | | | quantification of | |
| | | | | circulating | |
| | | | | microRNAs for dogs | |
| | | | | with congestive | |
| | | | | heart failure | |
| | | | | secondary to | |
| | | | | myxomatous mitral | |
| | | | | valve degeneration. | |
| | | | | American journal of | |
| | | | | veterinary research. | |
| | | | | 2018 Feb | |
| | | | | 1;79(2): 163-9. | |
| cfa-miR-206- | 7 | UGGAAUGUAAGGAAG | | Yang VK, Loughran | (14) Li, Qinghong, et al. |
| 3p | | UGUGUGG | | KA, Meola DM, Juhr | “Expression profiling of |
| | | | | CM, Thane KE, Davis | circulating microRNAs in |
| | | | | AM, Hoffman AM. | canine myxomatous |
| | | | | Circulating exosome | mitral valve disease.” |
| | | | | microRNA associated | International journal of |
| | | | | with heart failure | molecular sciences 16.6 |
| | | | | secondary to | (2015): 14098-14108. |
| | | | | myxomatous mitral | |
| | | | | valve disease in a | |
| | | | | naturally occurring | |
| | | | | canine model. | |
| | | | | Journal of | |
| | | | | extracellular vesicles. | |
| | | | | 2017 Dec | |
| | | | | 1; 6(1): 1350088. | |
| cfa-miR-320- | 8 | AAAAGCUGGGUUGAGA | Estrogen, | Ren, Xiao-Ping, et al. | |
| 3p | | GGGCGA | Hippo, TGF- | “MicroRNA-320 is | |
| | | | ß | involved in the | |
| | | | | regulation of cardiac | |
| | | | | ischemia/reperfusion | |
| | | | | injury by targeting | |
| | | | | heat-shock protein | |
| | | | | 20.” Circulation | |
| | | | | 119.17 (2009): 2357- | |
| | | | | 2366. | |
| cfa-miR- | 9 | UGAGGGGCAGAGAGC | Estrogen | Jung, SeungWoo, | |
| 423a-5p | | GAGACUUU | | and Amy Bohan. | |
| | | | | “Genome-wide | |
| | | | | sequencing and | |
| | | | | quantification of | |
| | | | | circulating | |
| | | | | microRNAs for dogs | |
| | | | | with congestive | |
| | | | | heart failure | |
| | | | | secondary to | |
| | | | | myxomatous mitral | |
| | | | | valve degeneration.” | |
| | | | | American journal of | |
| | | | | veterinary research | |
| | | | | 79.2 (2018): 163- | |
| | | | | 169. | |
| cfa-miR-499- | 10 | UUAAGACUUGCAGUGA | p53 | Qin, Han, et al. “The | |
| 5p | | UGUUU | | altered expression | |
| | | | | profile of microRNAs | |
| | | | | in cardiopulmonary | |
| | | | | bypass canine | |
| | | | | models and the | |
| | | | | effects of mir-499 on | |
| | | | | myocardial ischemic | |
| | | | | reperfusion injury.” | |
| | | | | Journal of | |
| | | | | translational | |
| | | | | medicine 11 (2013): | |
| | | | | 1-9. | |
| cfa-let-7b-5p | 11 | UGAGGUAGUAGGUUGU | AMPK, | Ro, Woong-Bin, et al. | Ro, Woong-Bin, et al. |
| | | GUGGUU | FoxO, | “Expression profile | “Identification and |
| | | | HIF1, | of circulating | characterization of |
| | | | Hippo, | MicroRNAs in dogs | circulating microRNAs |
| | | | MAPK, p53, | with cardiac | as novel biomarkers in |
| | | | TGF-β | hypertrophy: a pilot | dogs with heart |
| | | | | study.” Frontiers in | diseases.” Frontiers in |
| | | | | veterinary science 8 | veterinary science 8 |
| | | | | (2021): 652224. | (2021): 729929. |
| cfa-let-7e-5p | 12 | UGAGGUAGGAGGUUGU | FoxO, | Jung, SeungWoo, | |
| | | AUAGUU | MAPK, p53, | and Amy Bohan. | |
| | | | PI3K-Akt, | “Genome-wide | |
| | | | TGF-β | sequencing and | |
| | | | | quantification of | |
| | | | | circulating | |
| | | | | microRNAs for dogs | |
| | | | | with congestive | |
| | | | | heart failure | |
| | | | | secondary to | |
| | | | | myxomatous mitral | |
| | | | | valve degeneration.” | |
| | | | | American journal of | |
| | | | | veterinary research | |
| | | | | 79.2 (2018): 163-169. | |
| cfa-let-7i-5p | 13 | UGAGGUAGUAGUUUGU | FoxO, | Satoh, Mamoru, et | |
| | | GCUGUU | Hippo, | al. “A cellular | |
| | | | MAPK, p53, | microRNA, let-7i, is a | |
| | | | PI3K-Akt, | novel biomarker for | |
| | | | TGF-β | clinical outcome in | |
| | | | | patients with dilated | |
| | | | | cardiomyopathy.” | |
| | | | | Journal of cardiac | |
| | | | | failure 17.11 (2011): | |
| | | | | 923-929. | |
| cfa-miR-29a- | 14 | UAGCACCAUCUGAAAU | FoxO, | Dawson, Kristin, et | |
| 3p | | CGGUUA | Hippo, p53, | al. “MicroRNA29: a | |
| | | | PI3K-Akt | mechanistic | |
| | | | | contributor and | |
| | | | | potential biomarker | |
| | | | | in atrial fibrillation.” | |
| | | | | Circulation 127.14 | |
| | | | | (2013): 1466-1475. | |
| cfa-miR-486- | 15 | UCCUGUACUGAGCUG | | Wang, Hongyun, et | |
| 5p | | CCCCGAG | | al. “Percutaneous | |
| | | | | intracoronary | |
| | | | | delivery of plasma | |
| | | | | extracellular vesicles | |
| | | | | protects the | |
| | | | | myocardium against | |
| | | | | ischemia-reperfusion | |
| | | | | injury in Canis.” | |
| | | | | Hypertension 78.5 | |
| | | | | (2021): 1541-1554. | |
| (Normalizers) | | | | | |
| cfa-miR-17- | 16 | CAAAGUGCUUACAGUG | FoxO, | Yang, Vicky K., et al. | |
| 5p | | CAGGUAG | Hippo, | “Dysregulation of | |
| | | | MAPK, | valvular interstitial | |
| | | | mTOR, p53, | cell let-7c, miR-17, | |
| | | | PI3K-Akt, | miR-20a, and miR- | |
| | | | TGF-β, TNF, | 30d in naturally | |
| | | | Wnt | occurring canine | |
| | | | | myxomatous mitral | |
| | | | | valve disease.” PloS | |
| | | | | one 13.1 (2018): | |
| | | | | e0188617. | |
| cfa-miR- | 17 | CAGUGCAAUGAUGAA | FoxO, TGF- | Ro, Woong-Bin, et al. | |
| 130b-3p | | AGGGCAU | B | “Identification and | |
| | | | | characterization of | |
| | | | | circulating | |
| | | | | microRNAs as novel | |
| | | | | biomarkers in dogs | |
| | | | | with heart diseases.” | |
| | | | | Frontiers in | |
| | | | | veterinary science 8 | |
| | | | | (2021): 729929. | |
| cfa-miR-20a- | 18 | UAAAGUGCUUAUAGUG | ErbB, FoxO, | Yang, Vicky K., et al. | |
| 5p | | CAGGUAG | MAPK, | “Dysregulation of | |
| | | | PI3K-Akt, | valvular interstitial | |
| | | | TGF-ß, Wnt | cell let-7c, miR-17, | |
| | | | | miR-20a, and miR- | |
| | | | | 30d in naturally | |
| | | | | occurring canine | |
| | | | | myxomatous mitral | |
| | | | | valve disease.” PloS | |
| | | | | one 13.1 (2018): | |
| | | | | e0188617. | |
| cfa-miR-23a- | 19 | AUCACAUUGCCAGGGA | mTOR, p53 | Liu, Dishiwen, et al. | |
| 3p | | UUU | | “Cardiac fibroblasts | |
| | | | | promote ferroptosis | |
| | | | | in atrial fibrillation | |
| | | | | by secreting exo- | |
| | | | | miR-23a-3p targeting | |
| | | | | SLC7A11.” Oxidative | |
| | | | | Medicine and | |
| | | | | Cellular Longevity | |
| | | | | 2022 (2022). | |
| cfa-miR-26a- | 20 | UUCAAGUAAUCCAGGA | Hippo, p53, | Luo, Xiaobin, et al. | |
| 5p | | UAGGCU | Wnt | “MicroRNA-26 | |
| | | | | governs | |
| | | | | profibrillatory | |
| | | | | inward-rectifier | |
| | | | | potassium current | |
| | | | | changes in atrial | |
| | | | | fibrillation.” The | |
| | | | | Journal of clinical | |
| | | | | investigation 123.5 | |
| | | | | (2013): 1939-1951. | |

Data preparation and exploration: The Fireplex®-processed data set consisted of 97 miRNA expression profiles issued from 50 healthy controls and 47 MMVD cases. MMVD cases were further divided into 29 pre-clinical MMVD (stage B1/B2) and 18 clinical MMVD (stage C/D) cases. Data were generated in two batches of 48 and 49 samples, respectively. Each miRNA profile was formed of measuring the normalized mean fluorescence intensity (MFI) of 15 miRNAs common across the two data batches. For each miRNA profile, MFIs were standardised by a centred log-ratio transformation applied to each sample to handle the compositionality of the quantification of miRNA molecules derived from varying sequencing library sizes across samples (Fernandes, A. D. et al., Microbiome, 2, 15 (2014)). To manage variation due to batch effects, weighted PLS-DA-batch correction method was applied, as proposed in Wang 2023 (Yiwen Wang, Kim-Anh Lê Cao, Briefings in Bioinformatics, Volume 24, Issue 2, (2023). This method was specifically designed for an unbalanced batch x disease status setting. Two datasets were generated: (1) the first included 97 samples with the analysis focusing on characterisation and discrimination between healthy control and MMVD samples; (2) the second included only the MMVD samples and was used to explore potential markers that could discriminate pre-clinical (stage B1/B2) from clinical (stage C/D) MMVD cases. The two datasets were illustrated by heatmaps and approximately represented in two dimensions by principal component analysis (PCA) to allow initial exploration and identification of miRNA patterns. The first two principal components, accounting for the largest fraction of the original data variability, were used to produce a planar biplot display where samples and miRNA signals were jointly represented by points and rays from the origin respectively (with the rays indicating directions of increasing miRNA expression relative to the others).

Predictive classification modelling: After preliminary investigation and comparative assessment of alternative statistical and machine learning approaches to select an optimal predictive modelling formulation, penalised logistic regression (PLR) models (Park M Y, Hastie T., Biostatistics, 9 (1): 30-50 (2008); Hastie, T., et al., (2001), The Elements of Statistical Learning, Springer New York Inc., New York, NY, USA) were fitted to mean-centred data for the purpose of predictive classification of samples into (1) MMVD cases or healthy status; and (2) pre-clinical MMVD (stage B1/B2) or clinical MMVD (stage C/D). Formally, given a 2-class response variable y, taking values 1 (positive status) with probability p and 0 (negative status) with probability 1−p, and the vector of processed miRNA signals (and possibly other covariates) acting as predictors x=(x₁, . . . , xₚ), a logistic regression model of the form:

$$ln \frac{p}{1 - p} = {β}_{0} + {\sum }_{j = 1}^{p} {β}_{j} {x}_{j} .$$

was established. Using the maximum likelihood estimation method, PLR model coefficient estimates (\hat{β}₀, \hat{β}₁, . . . \hat{β}ₚ) were obtained by maximizing the penalized log-likelihood function:

$$max \{ {\sum }_{i = 1}^{n} [ {y}_{i} ( {β}_{0} + {\sum }_{j = 1}^{p} {β}_{j} {x}_{ij} ) - ln ( 1 + {e}^{{β}_{0} + {\sum }_{j = 1}^{p} {β}_{j} {x}_{ij}} ) ] - λ {\sum }_{j = 1}^{p} {\vert {β}_{j} \vert}^{2} \} ,$$

where n refers to the number of samples and λ is the penalty parameter, so that the coefficients less contributing to the prediction of the outcome were shrunk toward zero. Including such a penalty aids in preventing overfitting, favoring model unbiasedness, sparsity, and a stable fit with large numbers of predictors, typically affected by multicollinearity. Given the model estimates, the predicted status probabilities for a sample were obtained using:

$$\hat{p} = \frac{1}{1 + {e}^{- ( {\hat{β}}_{0} + {\hat{β}}_{1} {x}_{1} + … + {\hat{β}}_{p} {x}_{p} )}} ,$$

with a sample being allocated the status with the highest probability. Thus, in the MMVD group against healthy control setting, a sample was diagnosed positive when \hat{p}>0.5 and negative otherwise. For pre-clinical MMVD (stage B1/B2) against clinical MMVD (stage C/D), the latter was considered the positive status. Tuning to determine the level of shrinkage λ, parameter fitting by maximum likelihood, and performance assessment were all embedded into a 10-fold cross-validation (CV) pipeline. That is, the input data were randomly partitioned into ten folds, with nine folds used to train the model and one-fold used as validation set sequentially. This randomization was repeated 5 times. As the number of samples in each class was unbalanced in the input data, particularly when confronting pre-clinical MMVD (stage B1/B2) and clinical MMVD (stage C/D), 29 and 18 samples, respectively, the minority class was over-sampled within the CV runs by using the synthetic minority sampling technique (SMOTE) (Nitesh V. et al., J. Artif. Int. Res. 16; 1, 321-357 (2002)) aiming to minimize the effect of this issue on model performance assessment. Note that the sex of the animals was initially considered in the PLR models as potential predictor along with the miRNA signals, but the associated model coefficients \hat{β} resulted not statistically significant at the usual 5% significance level and it was then omitted from the final modelling based on miRNA signals only. Performance metrics included overall accuracy, area under the receiver operating curve (AUC-ROC), sensitivity, specificity and the F1 score (regarded as fairer assessment of accuracy in case of unbalanced classes) (Hastie, T. et al., (2001), The Elements of Statistical Learning, Springer New York Inc., New York, NY, USA). All these metrics ranged in [0,1], with values closer to 1 indicating better performance. They were measured against each validation set and averaged across CV runs to assess how the model might perform when asked to predict from independent blind samples. These measures were accompanied by 95% confidence intervals (CI) where available. The model training pipeline and all data analyses and graphical representations were set up and conducted on the R system for statistical computing v4.2.1 (R Core Team. R: A language and environment for statistical computing. R Foundation for Statistical Computing, Vienna, Austria).

### Example 1: Description and Classification of the Cohort Population

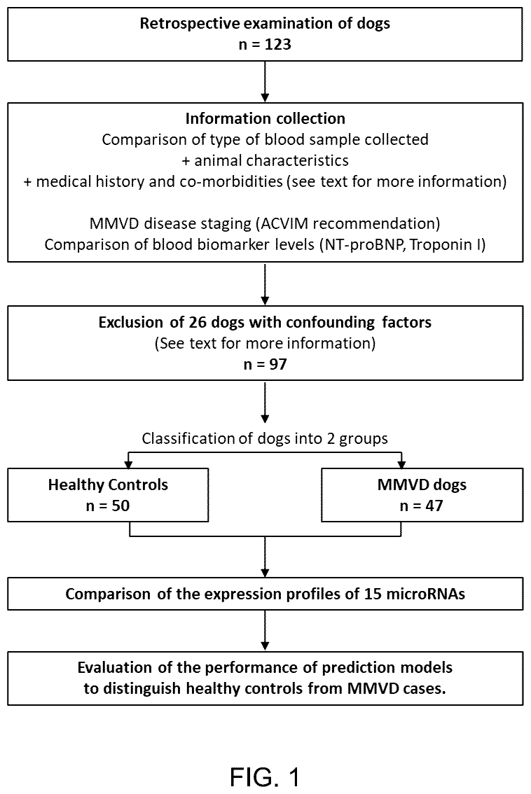

One hundred and twenty-three dogs were enrolled in the study. Twenty-six dogs were excluded because of clinically important systemic or other organ-related diseases, leaving 97 dogs in the final cohort (FIG. 1). Excluded cases in this cohort presented with the following: systemic hypertension (n=11), congenital heart disease (n=2), chronic kidney disease (n=2), dilated cardiomyopathy (n=2), pulmonic stenosis (n=2), significant pulmonary hypertension not associated with increased left atrial pressure (n=2), adrenal mass (n=1), Angiostronylosis (n=1), Cushing's disease (n=1), liver mass (n=1) and neuroendocrine tumour (n=1).

The 97 dogs included in the study were first divided into two groups: healthy controls (n=50) and MMVD cases (n=47) (Table 3). Within the 47 MMVD cases, 21 dogs were stage B1 (44.7%), 8 dogs were stage B2 (17%), 8 dogs were stage C (17%), and 10 dogs were stage D (21.3%).

For each dog included in the study, the type of blood sample collected as well as the age, sex, and breed of each animal were recorded (Table 3). Of the healthy cases, 26 samples (52%) were sera and 24 (48%) were plasma samples, while in the MMVD cases, 46 samples (97.9%) were sera and only 1 (2.1%) was a plasma sample. Statistically significant differences in the distribution of male and female animals were detected between healthy and MMVD cases (χ₁²=4.4628, p=0.0346).

TABLE 3
Characteristics of 97 dogs recruited to the study.

| | Healthy Controls | MMVD Dogs |
|---|---|---|
| Number of dogs | 50 | 47 |

| | | Number | % | Number | % |
|---|---|---|---|---|---|
| Sample type | Serum | 26 | 52.0 | 46 | 97.9 |
| | Plasma | 24 | 48.0 | 1 | 2.1 |
| Age | Median | 4.0 | | 10 | |
| | [Range] | [2.0-15.0] | | [6.1-13.8] | |
| Sex | F | 26 | 52.0 | 7 | 14.9 |
| | FN | 9 | 18.0 | 15 | 31.9 |
| | M | 10 | 20.0 | 9 | 19.1 |
| | MN | 5 | 10.0 | 16 | 34.0 |
| Breed | CKCS | 2 | 4.0 | 19 | 40.4 |
| | Chihuahua | 1 | 2.0 | 3 | 6.4 |
| | Standard Poodle | 1 | 2.0 | 0 | 0.0 |
| | Miniature Schnauzer | 1 | 2.0 | 0 | 0.0 |
| | Yorkshire Terrier | 0 | 0.0 | 2 | 4.3 |
| | Cocker Spaniel | 5 | 10.0 | 2 | 4.3 |
| | Shih Tzu | 0 | 0.0 | 1 | 2.1 |
| | Boston Terrier | 1 | 2.0 | 0 | 0.0 |
| | Mixed breed | 1 | 2.0 | 16 | 34.0 |
| | Other pure breeds | 38 | 76.0 | 4 | 8.5 |

### Example 2: NT-proBNP and Troponin I Levels

Results for at least one cardiac biomarker (NT-proBNP or cTnI) were available for a proportion (n=44) of the controls (n=17) and MMVD cases (n=27). The low number of available results meant statistical comparison of these diagnostics to the presented miRNA profiling was not possible, however the results and the percentage of cases correctly classified by NT-proBNP and/or cTnI, and the percent of this proportion of cases correctly classified by miRNA profiling are provided (Table 4).

TABLE 4
MMVD classification and blood biomarker levels in cases where available.

| | | | | Troponin I (cTnI) | | miRNA profiling | |
|---|---|---|---|---|---|---|---|
| | | NT-proBNP | | | Percent | Number | Percent |
| | | | Percent | | correctly | of dogs | correctly |
| | | | correctly | | classified | with NT- | classified |
| | | | classified as | Number of | as MMVD | proBNP | as MMVD |
| | | Number of | MMVD | dogs with | (>0.15 | and/or | with |
| | | dogs with | (>900 | available | or >0.07 | cTnI | miRNA |
| | Stage | available data | pmol/L) | data | ng/ml) | available | profiling |
| Healthy | | 15 | 100% | 17 | 94% | 17 | 88% |
| Controls | | | | | | | |
| MMVD | B1 | 12 | 33% | 9 | 33% | 16 | 94% |
| dogs | B2 | 2 | 50% | 4 | 0% | 4 | 100% |
| | C | 0 | NA | 4 | 75% | 4 | 100% |
| | D | 0 | NA | 3 | 100% | 3 | 100% |

### Example 3: Predictive Classification of Healthy Controls and MMVD Cases

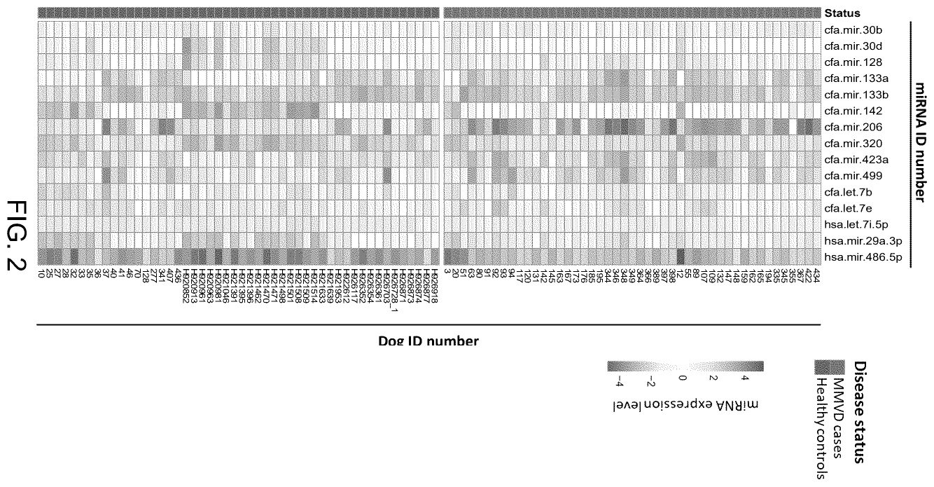

The miRNA expression profile of each of the 97 dogs included in the study were analyzed and represented as a heatmap (FIG. 2), distinguishing healthy from MMVD cases. The overall highest expression was observed for miRNA hsa-mir486-5p, particularly in the healthy control group. The lowest expression across all samples was observed for miRNA cfa-mir-206, particularly for MMVD samples.

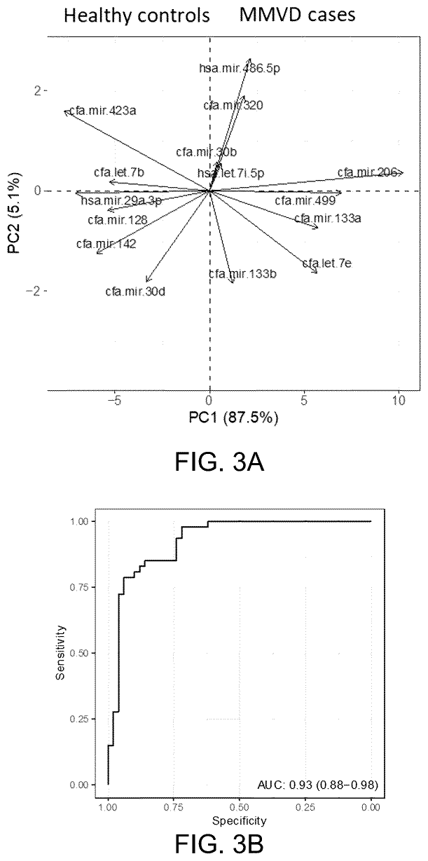

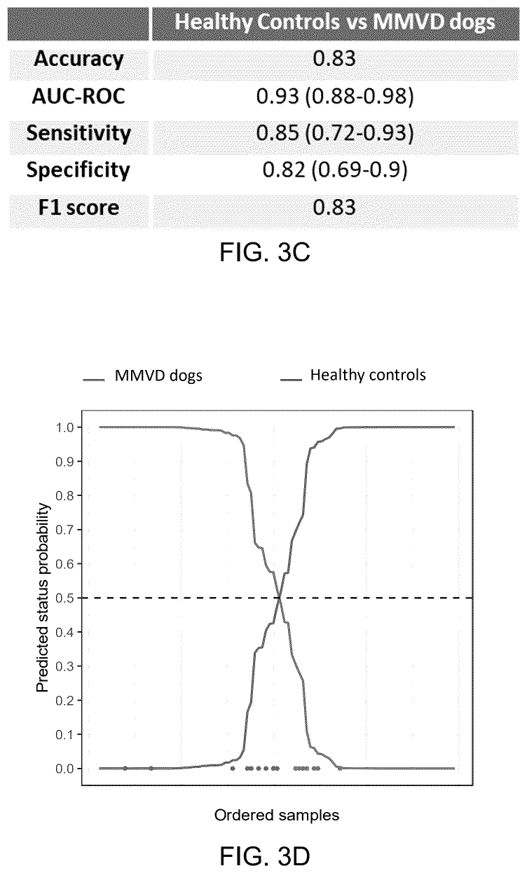

A summary of the performance of the prediction model fitted to distinguish between healthy controls and MMVD dogs is provided in FIG. 3. The PCA biplot of the entire miRNA dataset based on the first two principal components (PC1 and PC2) demonstrates that variability between MMVD and healthy samples was mostly represented along the first PC axis (PC1; 87.5% variation explained; FIG. 3A). Variation along PC1 is related to the differential expression of a small number of miRNAs, including cfa-mir-206-3p, the most highly expressed miRNA in the healthy group (right-hand side) and cfa-mir-29a-3p, the most highly expressed miRNA in the MMVD group (left-hand side). Other miRNAs, such as cfa-mir-133b-3p contributed to the variation in PC2 and was poorly associated with MMVD. Receiver operator characteristic analysis of the model provided an AUC value of 0.93 (0.88-0.98) (FIG. 3B), with an overall cross-validated accuracy of 0.83, a sensitivity of 0.85 (0.72-0.93), a specificity of 0.82 (0.69-0.90), and an F1 score of 0.83 (FIG. 3C). Misclassification of samples was more frequent for control than MMVD samples and occurred more often in samples assigned class probabilities close to 0.5 by the PLR model (FIG. 3D).

### Example 4: Predictive Classification of Pre-Clinical (Stage B) and Clinical (Stage C/D) MMVD Cases

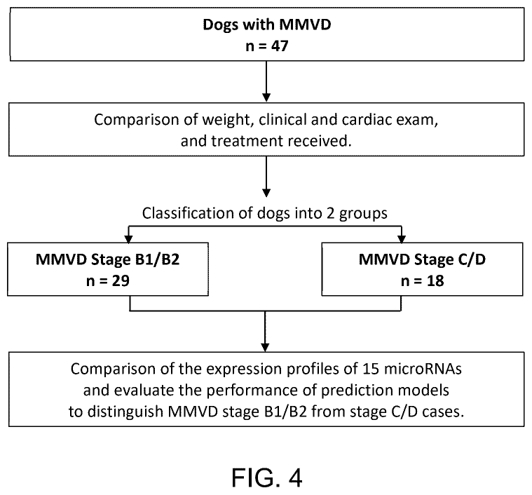

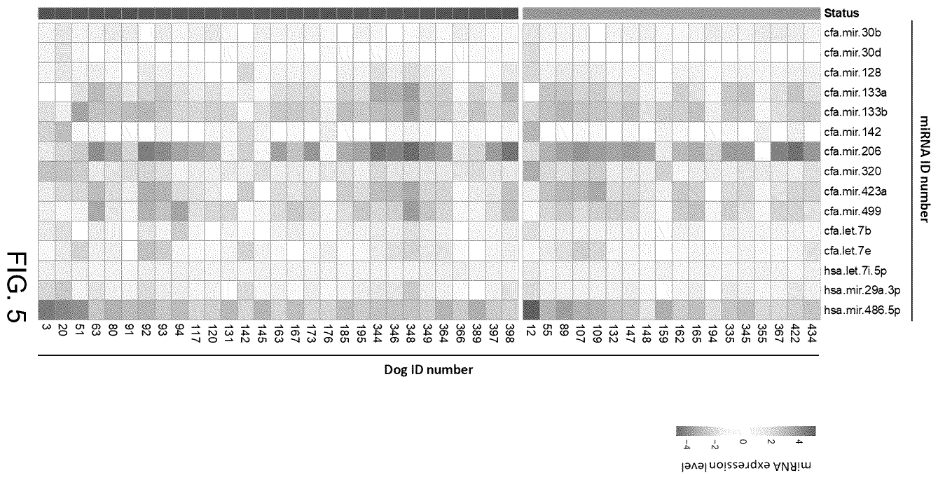

To assess classification of pre-clinical (stage B1/B2) from clinical (stage C/D) MMVD cases, the 47 MMVD dogs were subdivided into two groups (FIG. 4) based on clinical and cardiac examination by echocardiography, as per ACVIM guidelines (Keene, B W, et al., J Vet Intern Med., 33:1127-1140 (2019)). A total of 29 pre-clinical MMVD (stage B1/B2) cases, and 18 clinical MMVD (stage C/D) cases were identified (Table 5). The results of the clinical examination and echocardiographic measurements of the pre-clinical and clinical MMVD groups are summarized in Table 5. Importantly, the median weight of MMVD stage B1/B2 and stage C/D groups were highly comparable with 9.8 kg (from 3.4 to 42 kg) and 8 kg (from 2.6 to 31.5 kg), respectively. In keeping with clinical expectation, all echocardiographic measurements taken to evaluate cardiac function were higher in MMVD stage C/D than stage B1/B2 patients. In accordance with disease severity, few dogs in the MMVD stage B1/B2 group were receiving cardiac medications at the time of presentation (Table 5). In contrast, the majority of MMVD stage C/D dogs (16 dogs, 88.9%) were receiving a combination of Pimobendan, Benazepril, Spironolactone and either Furosemide or Torsemide, at the time of presentation.

TABLE 5
Weights, clinical and cardiac exams, and medication
of 47 MMVD dogs recruited to the study.

| | MMVD | MMVD |
|---|---|---|
| | Stage B1/B2 | Stage C/D |
| Number of dogs | 29 | 18 |

| | Median | Range | Median | Range |
|---|---|---|---|---|
| Weight (kg) | 9.8 | [3.4-42] | 8 | [2.6-31.5] |

| | | Median | IQR | Median | IQR |
|---|---|---|---|---|---|
| Clinical exam | Respiratory rate | 30 | 10.0 | 36 | 16.0 |
| | Heart rate | 120 | 38.0 | 135 | 30.0 |
| | Heart murmur grade | 3 | 2.0 | 5 | 0.0 |
| | (/6) | | | | |
| | SBP (mmHg) | 147.5 | 22.8 | 140 | 27.5 |
| Cardiac | VHS (RL thoracic | 10.0 | 1.5 | 12.5 | 0.8 |
| measurements | radiograph) | | | | |
| and function | LVIDdN (M-mode) | 1.5 | 0.2 | 2.1 | 0.4 |
| | LVIDsN (M-mode) | 0.9 | 0.2 | 1.0 | 0.5 |
| | FS % (M-mode) | 41.0 | 10.0 | 47.0 | 13.5 |
| | EDV (mLs) (SMOD) | 25.0 | 20.0 | 35.0 | 41.0 |
| | ESV (mLs) (SMOD) | 8.0 | 8.0 | 7.5 | 18.5 |
| | EDV/kg | 2.3 | 0.9 | 4.5 | 1.9 |
| | ESV/kg | 0.8 | 0.4 | 1.1 | 0.6 |
| | EF % (SMOD) | 63.5 | 9.2 | 72.5 | 14.3 |
| | LAmax (cm) | 3.3 | 1.0 | 4.5 | 1.2 |
| | LAmax/Ao annulus | 2.6 | 0.9 | 3.6 | 0.8 |
| | systole | | | | |
| | LA/Ao sh ax diastole | 1.5 | 0.2 | 2.2 | 0.4 |
| | Mitral E (m/s) | 0.7 | 0.3 | 1.1 | 0.6 |
| | Mitral E/IVRT | 1.2 | 0.6 | 2.2 | 1.6 |

| | | Number | % | Number | % |
|---|---|---|---|---|---|
| Treatment | Pimobendan | 5 | 17.2 | 16 | 88.9 |
| received at | Benazepril | 2 | 6.9 | 16 | 88.9 |
| time of initial | Spironolactone | 2 | 6.9 | 16 | 88.9 |
| assessment | Furosemide/ | 0 | 0.0 | 17 | 94.4 |
| | Torsemide | | | | |
| | Amlodipine | 0 | 0.0 | 1 | 5.6 |
| | Sotalol | 0 | 0.0 | 1 | 5.6 |

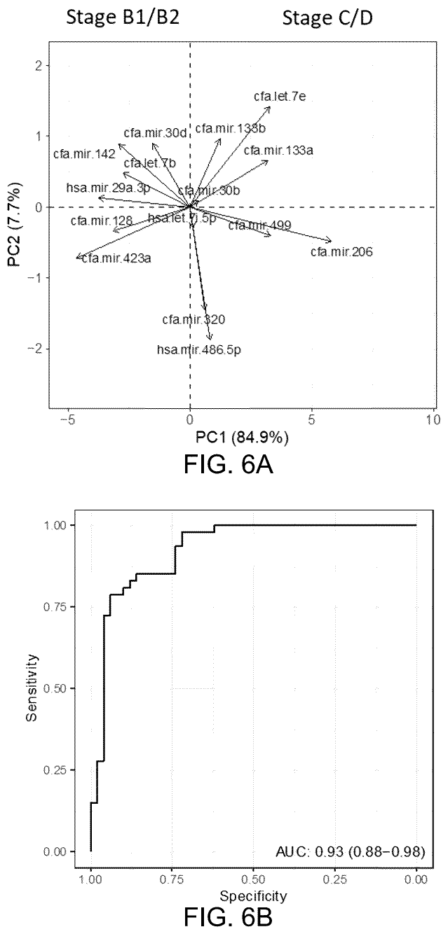

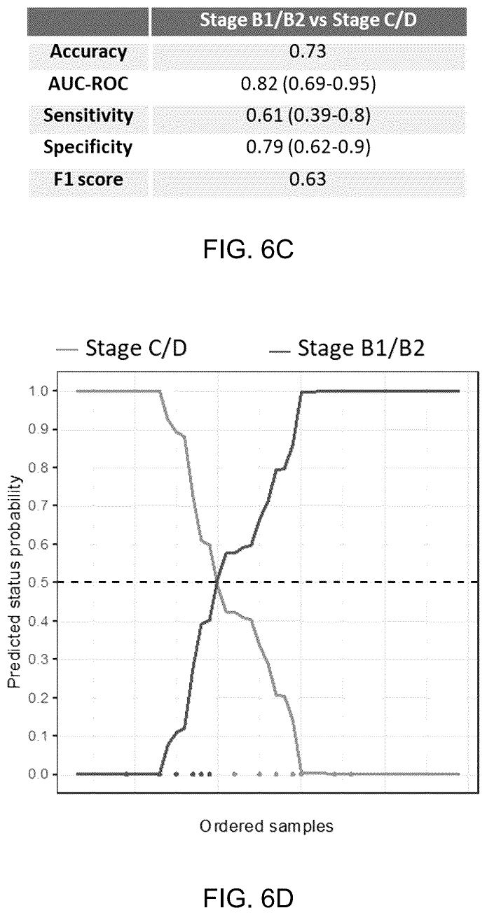

The miRNA expression profile of each of the 47 MMVD dogs was analyzed and represented as a heatmap (FIG. 5), distinguishing pre-clinical (stage B1/B2) and clinical (stage C/D) MMVD cases. As seen previously (FIG. 2), the highest expression was observed for miRNA hsa-mir486-5p, and lowest expression was observed for miRNA cfa-mir-206, however differentiation between pre-clinical and clinical MMVD samples was not as distinct. Groups defined by pre-clinical (B1/B2) versus clinical (C/D) stages are not well distinguished in the PCA biplot (FIG. 6A). The ROC analysis provided a moderately high AUC value of 0.82 (0.69-0.95), sensitivity of 0.61 (0.39-0.80) and specificity of 0.79 (0.62-0.90; FIGS. 6B and C). The F1 score was a moderate 0.63 in this case. The overall cross-validated accuracy was 0.73, with misclassification being similar for B1/B2 samples and C/D samples (FIG. 6D).

Among the 97 dogs included in the study, 50 dogs were classified as healthy controls and 47 were identified as MMVD cases. Following assessment of disease severity by the attending cardiologist MMVD cases were further divided from stage B1 to stage D including 21 dogs in stage B1 (44.7%), eight dogs were in stage B2 (17%), eight dogs were in stage C (17%), and 10 dogs were in stage D (21.3%).

The expression profiles of the selected 15 miRNAs allowed convincing distinction of healthy from MMVD cases in the study, with the optimized predictive model providing an AUC value of 0.93, with an overall cross-validated accuracy of 0.83, a sensitivity of 0.85 and a specificity of 0.82. Variability between MMVD and healthy samples was mostly related to differential expression of a small number of miRNAs including cfa-mir-206-5p, most highly expressed in the healthy group, and cfa-mir-29a-3p, most highly expressed in the MMVD group. cfa-mir-206-5p has previously been associated with atrial fibrillation and autonomic nerve remodeling in dogs (Zhang Y, et al., PLOS One. 27; 10 (3): e0122674 (2015)) something commonly encountered in MMVD although not present in the included cases of this study. The increased expression of cfa-mir-29a-3p has seen in MMVD cases and fits with observations in rat models in which increased levels of this marker were associated with cardiac hypertrophy and remodeling (Zhang Y, et al., PLOS One. 27; 10 (3): e0122674 (2015)). Other miRNAs such as cfa-mir-133b-3p were poorly associated with any group. This contradicts previously obtained observations in Dachshunds where miR-133b was down-regulated in Stage C compared with Stage A dogs and thus thought to be a possible marker of CHF. This discrepancy could be linked to variation in size and the wider range of breeds in the current study population.

One possible limitation of the study is some structuring in the data due to age. Although efforts were made to obtain a similar population in the control and MMVD groups, the median age of the healthy control group was lower (4 years) than the median age of the MMVD group (10 year), although the age range of both groups does overlap (2- to 15-year-old and 6.1- to 13.8-year-old, respectively). This is largely related to age being an important factor in MMVD making it difficult to design experiments to counter this. However, the possibility of an age-related effect in our data should be considered. Sex has also been reported as an important risk factor in MMVD (Borgarelli M, et al., J Vet Car-diol.; 6:27-34 (2004); Keene, B W, et al. J Vet Intern Med. 33:1127 1140 (2019)). With 35 females (52% intact and 18% neutered) and 15 males (20% intact and 10% neutered) in healthy controls, and 22 females (14.9% intact and 31.9% neutered) and 25 males (19.1% intact and 34% neutered) in MMVD dogs, the current study also concludes statistically significant differences in the distribution of sexes between healthy controls and MMVD dogs. However, sex was not identified as a significant factor of disease when included in the predictive models along with the miRNA profiles.

For the comparison of miRNA profiles between pre-clinical (stage B1/B2; n=29) from clinical (stage C/D; n=18) MMVD cases, expression patterns from the 47 MMVD dogs allowed some distinction between the two groups. ROC analysis suggested a moderately high AUC value of 0.82, with sensitivity of 0.61 and specificity of 0.79. The 95% confidence intervals for these metrics showed a marked variability across cross-validation runs, and the overall cross-validated accuracy was 0.73, with misclassification being here more frequent for B1/B2 samples than in stage C/D samples, and the F1 score was moderate with 0.63. Taken together, these data suggest a higher level of uncertainty in separating pre-clinical from clinical MMVD cases, compared to the comparison of control and MMVD cases. Such reduction in sensitivity and accuracy is likely related to the reduced number of samples within this comparative group and would likely be enhanced with an increased sample size.

In the MMVD group, there was a breed over-representation of Cavalier King Charles Spaniels (CKCS) (40.4%). Breed is an important risk factor for MMVD (Borgarelli M, et al., J Vet Car-diol.; 6:27-34 (2004); Keene, B W, et al. J Vet Intern Med. 33:1127 1140 (2019)) and the potential influence of specific breed miRNA expression profiles cannot be excluded. There is breed variation for NT-proBNP (Sjöstrand, K., et al., J Vet Intern Med 28 (2): 451-457, (2014); Gomart, S., et al., J Small Anim Pract 61 (6): 368-373 (2020); Misbach, C., et al.; Res Vet Sci 95 (3): 879-885 (2013)) and the same is possible for miRNA expression. However, formal statistical assessment and comparison between breeds and miRNA profiles were not possible in this study due a high diversity of breeds types providing a low number of individuals per breed. In the future, greater numbers of specific breeds with and without MMVD in different stages will allow to explore any breed influence further.

It was not possible to statistically compare miRNA results and conventional CBs, since there were only a low number of CB results available within the study. Subjectively in these cases, miRNA profiling performed comparably or better, especially in early-stage disease, but more detailed inferences cannot be made at present. With CBS, although there is a trend increasing values with stage of MMVD (Häggström, J., et al.; J Vet Cardiol 2 (1): 7-16 (2000); Wolf, J., et al.; Vet Clin Pathol 42 (2): 196-206 (2013)), there is no distinct cut-off for a specific stage of the disease (e.g. B2). Since progression of MMVD through the stages is a continuum, this is likely true for miRNA expression. However, the use of a panel of markers gives rise to the possibility of producing a fingerprint profile at each stage, with the potential to identify individual or patterns of miRNA expression which may be predictive of impending stage progression. It is anticipated that forthcoming serial, longitudinal monitoring of cases will provide further insight into this possibility.

Although limited in scope, comparison of the diagnostic capability of miRNA profiling to existing NT-proBNP and cTnI biomarkers showed strong promise. The miRNA profiling method performed well in classifying controls correctly, showed an apparent advantage in accurate detection of early-stage B1 and B2 cases over the existing biomarkers, and was comparable in stage C and D cases (Table 5). This provides potential for an improved method of early disease diagnosis, following further validation with a larger sample cohort.

In summary, this study provides strong evidence that a multiplexing detection assay of blood miRNAs can be a useful diagnostic tool for identification of MMVD cases in canine populations. Additionally, miRNA profiling provides the possibility to differentiate between pre-clinical (stage B1/B2) and clinical MMVD (stage C/D). This predictive modelling analysis would now benefit from training on a larger canine cohort constituted of a variety of MMVD cases to continually improve the probabilistic classification algorithms. MicroRNA diagnostic technology has great upcoming potential not only in the field of veterinary cardiology, but current and future applications within the broader veterinary sphere. This technology is patent pending.

### Example 5: miRNA Profiling with Machine Learning Optimisation: Equine MR

### Materials and Methods

Before analysis, the percentage identity and sequence coverage of homologous miRNAs were assessed by reciprocal BLAST using the canine miRNA sequences. Cardiac panel miRNA profiles from 154 samples (77 controls and 77 MR cases) collected between 2015-2023 were normalized in Fireplex using previously identified control miRNAs. Data were standardized and Yeo Johnson transformed. Fourteen machine learning algorithms previously used on canine data were trained with 10-fold cross validation across five repeats. Model classification accuracy was assessed using ROC AUC, overall accuracy, Cohen's kappa, sensitivity, and specificity.

### Results

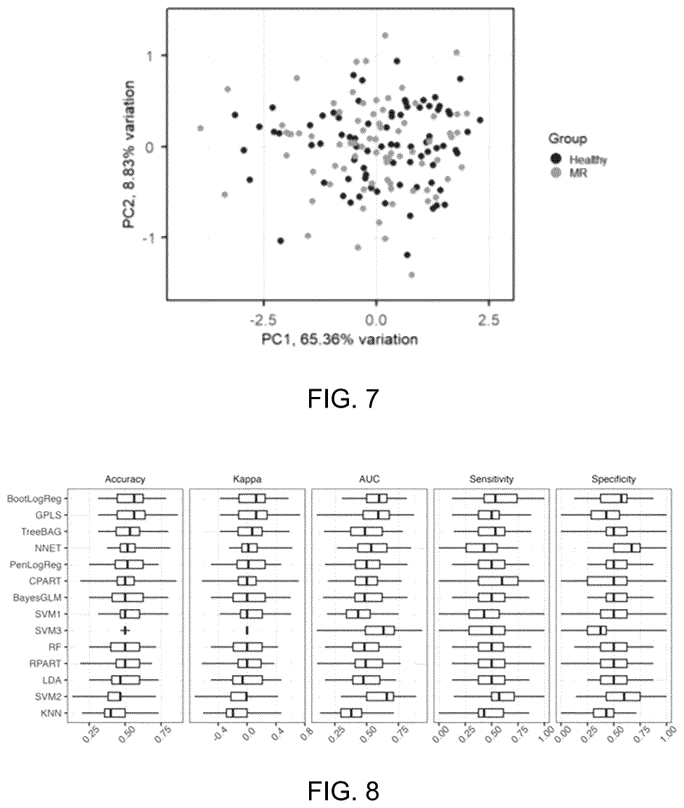

There was broad shared identity between miRNAs included on the canine and feline panel, with only cfa-miR-320-3p, cfa-let-7b-5p, and cfa-let-7i-5p having less than 100% identity in equines (Table 6). MicroRNA profiles of controls and MR samples initially have limited obvious separation on the first five axes of a principal component analysis (PCA) (The first two axes are shown in FIG. 7).

However, following machine learning analysis the most accurate model for binary classification (control vs MR) was boosted logistic regression (BootLogReg), with an overall accuracy 56%, 0.60 ROC AUC, sensitivity 0.58 and specificity 0.57 (FIG. 8). This indicates that the current panel has discriminatory power for equine MR.

TABLE 6
Sequence identity of miRNAs to equines.

| SEQ | | | Identity to reference |
|---|---|---|---|
| ID NO: | miRNA | Mature Sequence | E. caballus miRNA |
| 1 | cfa-miR-30b- | UGUAAACAUCCUACACUCAGCU | eca-miR-30b (100% |
| | 5p | | identity) |
| 2 | cfa-miR-30d- | UGUAAACAUCCCCGACUGGAAGCU | eca-miR-30d (100% |
| | 5p | | identity) |
| 3 | cfa-miR-128- | UCACAGUGAACCGGUCUCUUU | eca-miR-128 (100% |
| | 3p | | identity) |
| 4 | cfa-miR- | UUGGUCCCCUUCAACCAGCUGU | eca-miR-133a (100% |
| | 133a-3p | | identity) |
| 5 | cfa-miR- | UUUGGUCCCCUUCAACCAGCUA | eca-miR-133b (100% |
| | 133b-3p | | identity) |
| 6 | cfa-miR-142- | CCCAUAAAGUAGAAAGCACUA | eca-miR-142-5p |
| | 5p | | (100% identity) |
| 7 | cfa-miR-206- | UGGAAUGUAAGGAAGUGUGUGG | eca-miR-206 (100% |
| | 3p | | identity) |
| 8 | cfa-miR-320- | AAAAGCUGGGUUGAGAGGGCGA | eca-miR-193b (80% |
| | 3p | | identity) |
| 9 | cfa-miR- | UGAGGGGCAGAGAGCGAGACUUU | eca-miR-423-5p |
| | 423a-5p | | (100% identity) |
| 10 | cfa-miR-499- | UUAAGACUUGCAGUGAUGUUU | eca-miR-499-5p |
| | 5p | | (100% identity) |
| 11 | cfa-let-7b-5p | UGAGGUAGUAGGUUGUGUGGUU | eca-let-7c (95% |
| | | | identity) |
| 12 | cfa-let-7e-5p | UGAGGUAGGAGGUUGUAUAGUU | eca-let-7e (100% |
| | | | identity) |
| 13 | cfa-let-7i-5p | UGAGGUAGUAGUUUGUGCUGUU | eca-let-7g (90% |
| | | | identity) |
| 14 | cfa-miR-29a- | UAGCACCAUCUGAAAUCGGUUA | eca-miR-29a (100% |
| | 3p | | identity) |
| 15 | cfa-miR-486- | UCCUGUACUGAGCUGCCCCGAG | eca-miR-486-5p |
| | 5p | | (100% identity) |

Normalizers

| | | | |
|---|---|---|---|
| 16 | cfa-miR-17- | CAAAGUGCUUACAGUGCAGGUAG | eca-miR-17 (100% |
| | 5p | | identity) |
| 17 | cfa-miR- | CAGUGCAAUGAUGAAAGGGCAU | eca-miR-130b (100% |
| | 130b-3p | | identity) |
| 18 | cfa-miR-20a- | UAAAGUGCUUAUAGUGCAGGUAG | eca-miR-20a (100% |
| | 5p | | identity) |
| 19 | cfa-miR-23a- | AUCACAUUGCCAGGGAUUU | eca-miR-23a (100% |
| | 3p | | identity) |
| 20 | cfa-miR-26a- | UUCAAGUAAUCCAGGAUAGGCU | eca-miR-26a (100% |
| | 5p | | identity) |

### Example 6: Predictive Classification of Pre-Clinical (Stage B) and Clinical (Stage C/D) MMVD Cases

### Materials and Methods

The panel of miRNA profiles for MMVD, DCM and Control cases was jointly normalized in Fireflex and preprocessed, including correction for batch effects, for subsequent predictive modelling. A curated collection of 15 statistical and machine learning classification models were trained on the panel and their relative performances were assessed based on (a) overall accuracy, kappa metric, area under the ROC, sensitivity and specificity for binary classification and (b) accuracy and kappa metrics for 3-group classification, with the results being averaged over 10-fold cross-validation runs repeated 5 times, with group imbalance handled by the SMOTE method where required.

### Results

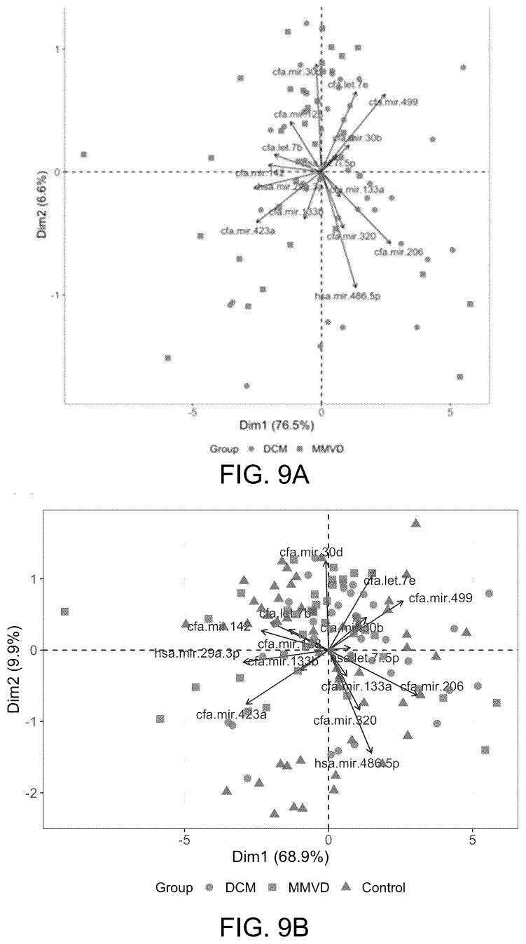

Two separate analyses were conducted: (a) distinction between MMVD and DCM cases and (b) distinction between MMVD, DCM and Control cases. Initial exploration shows in FIG. 9A some separation between MMVD and DCM miRNA profiles along the first axis in low-dimensional data representation via principal component analysis (PCA) biplot display (83.1% original variance explained). Analogously, FIG. 9B shows the PCA biplot analysis for the 3-group case (78.8% original variance explained). miRNA markers such as hsa.mir.486.5p, cfa.mir.206 or cfa.mir.128 appear to be most implied in the distinction between groups, particularly between DCM and MMVD cases.

The most accurate models for DCM vs. MMVD (Table 7) and DCM vs. MMVD vs. Control classifications (Table 8) were based on neural networks according to the current data, with summary performance statistics shown in Tables 7 and 8. High levels of discrimination between groups are obtained for case (a) (90% accuracy), with the largest rate of errors observed for MMVD cases being classed as DCM (7%); whereas moderate overall resolution is observed for case (b) (71% accuracy), with the largest contribution to errors observed between groups DCM and Control (15.8% error).

TABLE 7
Performance statistics from top predictive model
for binary classification into DCM and MMVD classes.
DCM vs. MMVD

| Stat | Mean |
|---|---|
| Accuracy | 0.9096 |
| Kappa | 0.8185 |
| ROC | 0.91 |
| Sens | 0.955 |
| Spec | 0.8633 |

| % | Reference | |
|---|---|---|
| Prediction | DCM | MMVD |
| DCM | 47.8 | 7 |
| MMVD | 2.2 | 43 |

| No. samples | |
|---|---|
| DCM | MMVD |
| 37 | 37 |

TABLE 8
Performance statistics from top predictive model for binary
classification into DCM vs. MMVD vs. Control classes.
DCM vs. MMVD vs. Control

| Stat | Mean |
|---|---|
| Accuracy | 0.71 |
| Kappa | 0.5626 |

| % | Reference | | |
|---|---|---|---|
| Prediction | DCM | MMVD | Control |
| DCM | 24.1 | 1.9 | 5.1 |
| MMVD | 2.2 | 20.5 | 9.8 |
| Control | 3 | 7 | 26.3 |

| No. samples | | |
|---|---|---|
| DCM | MMVD | Control |
| 37 | 37 | 52 |

The current cardiac miRNA panel provides high resolution to distinguish between DCM and MMVD cases using machine learning algorithms. Overall discrimination when a Control group is included is moderate, with the largest relative error being observed between DCM and control groups.

### Example 7: Comparison of microRNA Profiles of Dogs with Dilated Cardiomyopathy And Healthy Controls

### Materials and Methods

Study design and population: This was an exploratory, prospective, case control study using PICO methodology (Luijendijk H J. BMJ Evid Based Med. 2021 Mar. 31; 26 (4): 155-7) to phrase the research question. Ethical approval for this study was provided by the ethics committee of the University of Liverpool (VREC851) and Scotland's Rural College (SRUC).

Dogs were included in the study as cases if a diagnosis of DCM was made following clinical and cardiac evaluation (including echocardiography) by a cardiology specialist or a cardiology resident working under direct supervision of a cardiology specialist. Dogs were excluded from the study if no blood sample was available or if significant co-morbidity was evident. Dogs were included in the study as healthy controls if either clinical and cardiac evaluation (including echocardiography) by a cardiology specialist were unremarkable (University of Liverpool) or if a hematology and biochemistry profile was unremarkable and implied good general health (e.g. pre-anesthetic screening) and no cardiac murmur or arrhythmia was noted to be auscultated (SRUC).

Surplus serum or plasma blood samples (i.e. not collected for the purposes of this study) were utilized for microRNA analysis and collected between September 2020 and December 2021.

Clinical assessment of DCM cases: Dogs with dilated cardiomyopathy were recruited following referral for cardiac evaluation by the cardiology service at the University of Liverpool. Some were clinical cases, with presenting signs such as coughing, shortness of breath, lethargy etc. Others presented for DCM screening, or were asymptomatic, but the primary care veterinarian had detected a heart murmur or arrhythmia. A thorough history, including diet history, was recorded, to exclude nutritional DCM. A careful physical examination was performed, with special attention to the cardiovascular system. Heart rate and rhythm, respiratory rate and the findings of cardiac and pulmonary auscultation were recorded. The presence, point of maximal intensity and grade (0-6/6) of any heart murmur/s were noted, noting radiation. Systolic blood pressure was measured routinely in all clinical cases using Doppler sphygmomanometry (Acierno M J, Brown S, et al., J Vet Intern Med. 2018 November; 32 (6): 1803-1822). Two-dimensional, M-mode, color flow and spectral Doppler echocardiography was performed (as previously described (Palarea-Albaladejo J, et al., Front Vet Sci. 2024 Nov. 1; 11:1443847) using a GE Vivid E95 machine and a 6S (2.7-8.0) MHz or M5S (1.4-4.6 MHz) phased-array transducer and simultaneous single-channel ECG recording. Echocardiographic findings supporting a diagnosis of a DCM phenotype included diastolic and systolic volumes (based on one-plane Simpson's method of discs measured from right parasternal long axis 4 chamber views, which optimized left ventricular length and area), which both exceeded breed specific reference ranges (where available, e.g. Wess G, et al., J Vet Cardiol. 2017 October; 19 (5): 405-415, Stephenson H M, et al., J Vet Intern Med. 2012 September-October; 26 (5): 1140-7) or weight-based reference ranges (defined in diastole of >3.0 mLs/kg (Visser et al. 2019) and in systole of >1.50 mLs/kg (Wess G. J Vet Cardiol. 2022 April; 40:51-68)). Left ventricular M-modes were obtained by placing the cursor on the right parasternal short axis view, at level of chordae tendinae, bisecting the left ventricular cavity. Measurements used a leading edge-leading edge method, and both diastolic and systolic measurements had to exceed breed specific reference ranges where available, or weight-based reference ranges based on allometric scaling (Visser L C, et al., J Vet Intern Med. 2019 September; 33 (5): 1909-1920, Esser L C, Borkovec M, Bauer A, Häggström J, Wess G. J Vet Intern Med. 2020 November; 34 (6): 2242-2252). Simpson's derived ejection fraction and M-mode fractional shortening were noted. The geometrical changes in DCM, with rounding of the chamber were noted (determined as diastolic left ventricular length divided by the diastolic M-mode LV diameter; the sphericity index). Mitral valve M-mode E point to septal separation was measured. An important part of the echocardiographic study was to identify other congenital or acquired cardiac disease, which may result in the phenotype; such cases were excluded. However, some nodular thickening of the mitral valve and a central jet of mitral regurgitation was accepted in older dogs, as long as myxomatous degenerative changes were not concluded to be the primary pathology. Similarly, mild valvular regurgitation of other valves was not an exclusion criterion if not the primary pathology. Some dogs may have had equivocal echocardiographic findings, such as impaired left ventricular systolic function but without both diastolic and systolic left ventricular dilatation. These cases were excluded, but were serially monitored for progression to meet DCM inclusion criteria in the future (Bonagura J D, Visser L C. J Vet Cardiol. 2022 April; 40:15-50).

From left apical four chamber view, to assess diastolic function and estimate left sided filling pressures, the velocities of the transmitral E and A waves were documented, with the E/A ratio calculated, for animals in sinus rhythm. The isovolumic relaxation time (IVRT) was determined by documenting ventricular transmitral and left ventricular outflow tract flow on a left apical five-chamber view, with the time between end of left ventricular outflow tract flow to onset of the transmitral E wave. The mitral E/IVRT ratio was an estimate of left sided filling pressures (>1.8 considered abnormal in DCM; Schober K E, et al., J Vet Intern Med. 2010 November-December; 24 (6): 1358-68). The left atrium was also measured, with size indicated by systolic left atrial maximal diameter divided by the aortic annulus diameter (Strohm L E, et al., J Vet Cardiol. 2018 October; 20 (5): 330-342), and in short axis view in early diastole, by the 2D LA/Ao ratio (Hansson K, et al., Vet Radiol Ultrasound. 2002 November-December; 43 (6): 568-75).

If thoracic radiographs were taken, the vertebral heart size was measured from the right lateral view (Buchanan J W, Bücheler J. J Am Vet Med Assoc. 1995 January 15; 206 (2): 194-9) and any cardiogenic pulmonary oedema or effusions were noted. The current medication of these recruited cases was noted. In all patients not in sinus rhythm, a six or ten-lead electrocardiogram was obtained, to document any arrhythmia (e.g. atrial fibrillation, ventricular or supraventricular ectopy). In particular, in dogs with a tachyarrhythmia, the possibility of tachycardia induced cardiomyopathy, which may have resulted in the DCM phenotype, was considered, resulting in exclusion of affected patients. To evaluate this further, some DCM cases underwent 24-hour ambulatory ECG (Holter) monitoring.

General health screening with hematology, biochemistry and thyroid function testing was carried out in cases, as clinically indicated. Dogs with significant systemic conditions or which were hypothyroid prior to treatment of hypothyroidism for at least 2 months, were excluded from the DCM and the control groups. The cardiac biomarkers, either NT-pro-BNP or Troponin I (standard or high sensitivity assays) or both were assessed from blood samples in some cases and some controls, and data retrieved if available.

A subsection of the control group also underwent these investigations.

The diagnosis of dilated cardiomyopathy was defined based on echocardiography, as left ventricular dilatation in both diastole and systole, with impaired left ventricular systolic function with active exclusion of other systemic and cardiac conditions which may result in the DCM phenotype (Dukes-McEwan J, J Vet Cardiol. 2003 November; 5 (2): 7-19). The diagnosis of arrhythmogenic cardiomyopathy was defined in a predisposed breed as having significant ventricular arrhythmias (Wess G. J Vet Cardiol. 2022 April; 40:51-68, Wess G, et al., J Vet Cardiol. 2017 October; 19 (5): 405-415) in the absence of systemic causes of the arrhythmias. This subpopulation may or may not have had echocardiographic abnormalities.

Sample processing and selection of microRNA markers: Cases and controls were recruited between September 2020 and December 2021. After collection the blood samples were centrifuged to extract plasma or serum and stored in plain tubes at −20° C. for up to 10 months prior to analysis.

A novel panel utilizing 23 microRNA markers (15 informative, five normalizers, three off species controls) were previously developed by this team for diagnosis of MMVD (Palarea-Albaladejo J, et al., Front Vet Sci. 2024 November 1; 11:1443847). In this study for each dog 50 μl of blood or sera were used to analyze the expression profile for this same panel of markers following methods as previously described (Palarea-Albaladejo J, et al., Front Vet Sci. 2024 Nov. 1; 11:1443847). In brief each sample was incubated with microbead particles (Fireplex capture) specific to the 23 miRNA targets and processed with optimized hybridization (39° C.), melt off (62° C.) and capture temperatures (39° C.). MicroRNA expression was quantified by measuring the mean fluorescence intensity of microbead particles specific to each microRNA sequence. Expression values were normalized (‘geomean’ function; Fireplex Analysis Workbench 2.0.274, Abcam, Cambridge, UK).

Analysis of microRNA profiles of dogs with dilated cardiomyopathy compared to healthy controls: Data preprocessing of the miRNA expression profiles included scaling by centered log-ratio transformation (clr; i.e. for each sample, the values of the miRNA signals were each divided by the geometric mean of them all and log-transformed to facilitate comparability across samples of varying sequencing library size (Fernandes et al., 2014), correction of batch effects by the weighted PLS-DA-batch method (Wang Y, Lê Cao K A. 2023 Mar. 19; 24 (2): bbac622), and mean-centering. A range of supervised classification algorithms were preliminarily tested and benchmarked aiming to maximize the ability to discriminate between DCM and healthy control samples as the main objective of the study. Amongst a few alternatives providing comparable performance, the k-nearest neighbours (KNN) algorithm (Cover, Thomas, and Peter Hart. IEEE transactions on information theory 13.1 (1967): 21-27) stood slightly out in terms of overall accuracy and was chosen for this report.

In brief, the KNN algorithm is a model-free computational procedure that operates by identifying in the data a set of k closest samples to a given sample, using the Euclidean distance as proximity metric, and then assigning this latter to the most frequent predicted group within such neighborhood as a prediction. It was applied here to produce a binary classifier with the 15 preprocessed miRNA markers as predictors. The algorithm was set up within a cross-validation (CV) resampling scheme, so that the given collection of labelled samples was repeatedly (5 times) partitioned into 10 folds at random (5-time repeated 10-fold cross-validation). For each partition, and sequentially, 9 folds were used to inform the algorithm (training set) and the remaining one (validation set) was used to evaluate its predictive performance against the actual group label of the samples therein. Overall performance of the KNN algorithm was quantified by averaging results across CV runs. This generally produces a more realistic assessment than crude metrics from the entire data set being used as training set, since by CV the performance is evaluated on samples not involved in the training of the algorithm. Moreover, an optimal value of the tuning parameter k was determined within the own CV resampling process, aiming to maximize accuracy in classification (k 5 in this case after testing values in [1, 20]). Finally, note that the synthetic minority sampling technique (SMOTE) (Chawla, Nitesh V., et al. Journal of artificial intelligence research 16 (2002): 321-357.) was applied across CV runs to manage unbalance between diagnostic groups in the collected data (n=37 DCM and n=52 control cases). The method was implemented using computer routines of the package caret v6.0-94 on the R system for statistical computing v4.4.1 (R Core Team, 2021).

Four common metrics were considered to evaluate the performance of the KNN binary classifier: sensitivity, specificity, area under the receiver operating curve (AUC-ROC) and overall accuracy. These were computed using routines in the R packages MLeval and pROC (Robin et al., 2011) were accompanied by 95% confidence intervals (CI) where available. For all measures, higher values closer to 1 indicate better performance. For an approximate visual illustration of the results, the predictors were nonlinearly projected into low dimensional space using the uniform manifold approximation and projection (UMAP) method (McInnes et al., 2018) and used to produce a two-dimensional prediction grid.

A tentative rank of predictors according to their contribution to distinguish between groups was estimated based on the collected data. Note that the clr transformation implies that they reflect the contribution of the miRNA signals relative to the per-sample geometric average of the signals in the selected miRNA panel. The method used (Fisher et al., 2019) involved repeated random permutations of the values of the predictors, through extensive resampling from the data (here 1000 permutation rounds on each predictor), and assessment of the loss in AUC-ROC (1-AUC loss function) when a predictor was removed.

Moreover, aiming to form a diseased group with richer information to be contrasted with the healthy control group (n=52), the same approach was applied combining the available miRNA profiles from MMVD cases with those from DCM cases to form a joint diseased group (n=74). The CV resampling procedure again set an optimal value of k=5 for the KNN classifier here. Finally, even if the group sizes were limited (n=37 in both cases), the possibility of finer-grain differentiation between DCM and MMVD cases was explored. Note that for this latter analysis: (1) no SMOTE adjustment was implemented given the balanced group sizes; and (2) a value k=7 was determined for the KNN parameter.

### Results

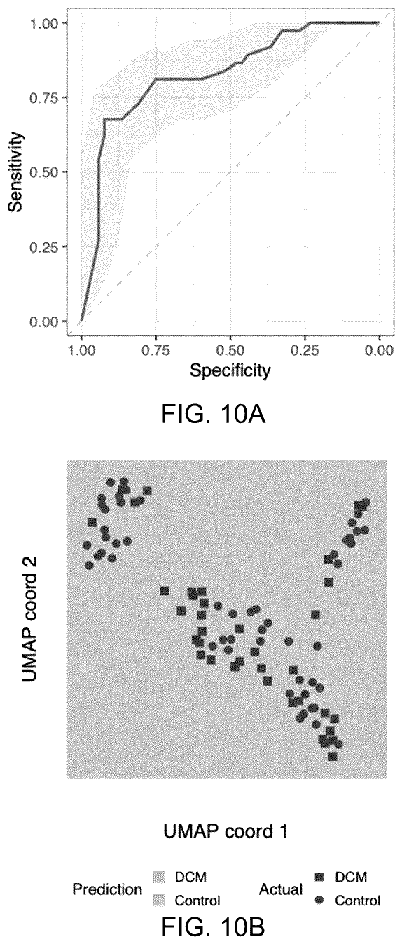

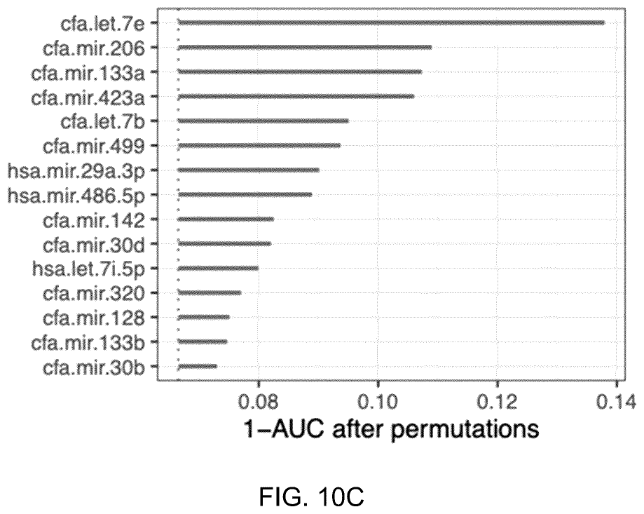

Table 9 summarizes the performance of the deployed platform for DCM diagnosis from the curated panel of blood miRNA profiles. The top shows the cross-validated confusion matrix obtained using the KNN algorithm to discriminate between DCM and healthy control samples. This confronts predicted group as provided by the method against group predetermined by gold standard cardiac evaluation (cell entries are percentual average counts across CV resampling runs). The bottom shows the associated performance metrics averaged across CV resampling runs. Misclassification occurred more frequently for healthy samples, according to the reference cardiac evaluation, being assigned the DCM group (15.73% overall). On the other hand, about 7.87% of DCM cases were unidentified by the microRNA-based platform. The averaged overall accuracy was 0.76 and the ROC analysis resulted in a moderately high AUC value of 0.83 (95% CI: 0.74-0.92). FIG. 10A shows the ROC curve of the miRNA-based classifier, including estimated 95% confidence bands. It can be observed that the curve is significantly distant from the random classifier (dashed diagonal line). FIG. 10B illustrates approximately the nonlinear decision boundaries of the classifier in a 2-dimensional prediction grid using UMAP coordinates derived from the preprocessed miRNA profiles. The points indicate the actual group membership of the samples. Finally, FIG. 10C provides the estimated ranking of predictors according to their relative contributions to distinguish between DCM and healthy samples (most relevant at the top, least relevant at the bottom). The lengths of the bars represent mean values of the importance metric over permutations for each predictor.

TABLE 9
Performance of microRNA-based platform to distinguish between
dilated cardiomyopathy and healthy control animals: cross-validated
confusion matrix (top) and performance metrics (bottom).

| | | Gold standard cardiac evaluation | |
|---|---|---|---|
| | | DCM yes | DCM no |
| MicroRNA cardiac | DCM yes | 33.71% | 15.73% |
| platform | DCM no | 7.87% | 42.7% |

| Performance metric | Cross-validated value [95% confidence interval] |
|---|---|
| Sensitivity | 0.81 [0.88-0.91] |
| Specificity | 0.73 [0.60-0.83] |
| AUC-ROC | 0.83 [0.74-0.92] |

### Example 8: Comparison of microRNA Profiles of Dogs with Cardiac Disease (Dilated Cardiomyopathy and Myxomatous Mitral Valve Disease) with Normal Controls

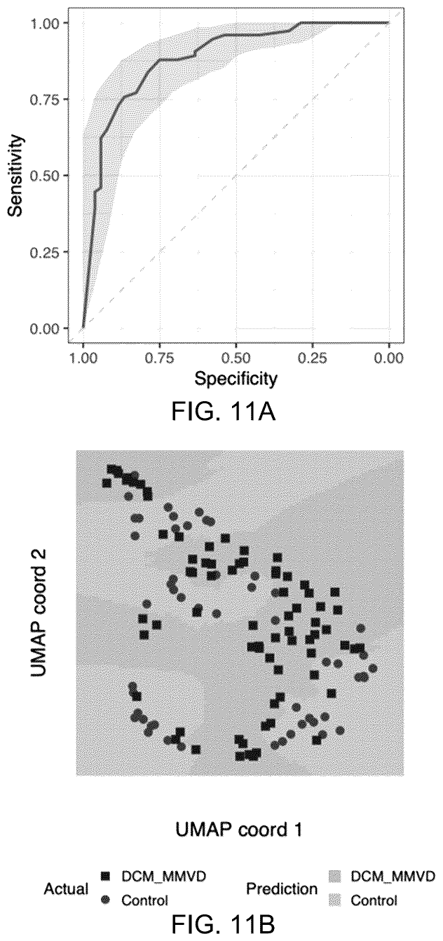

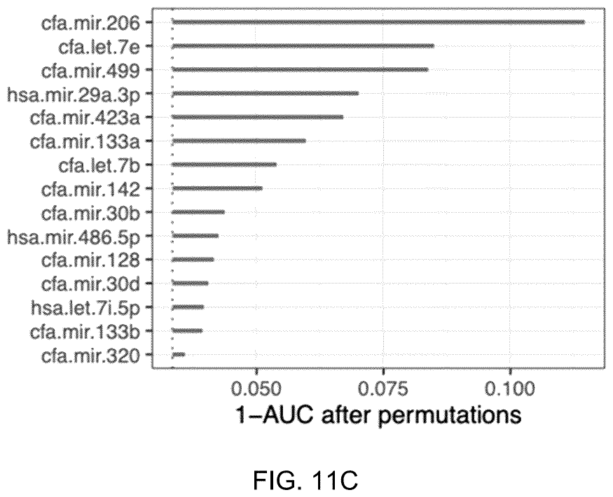

Here we considered the augmented group combining DCM and MMVD samples (n=74) against the healthy control group (n=52), which involved a more balanced comparison between diseased and healthy animals. The KNN classifier retrained on this basis reached improved performance in all common performance metrics relative to the previous analysis based on DCM only, with overall accuracy of 0.82 and a more precise AUC of 0.88 (95% CI: 0.82-0.94) (see Table 10 for a summary). Analogously, FIG. 11 shows the corresponding ROC curve, approximate prediction grid in low dimensions, and the resulting ranking of predictors.

TABLE 10
Performance of microRNA-based platform to distinguish
between diseased (DCM and MMVD cases combined) and
healthy control animals: cross-validated confusion
matrix (top) and performance metrics (bottom).

| | | Gold standard cardiac evaluation | |
|---|---|---|---|
| | | Cardiac disease | Healthy control |
| MicroRNA cardiac | Cardiac disease | 49.21% | 8.73% |
| platform | Healthy control | 9.52% | 32.54% |

| Performance metric | Cross-validated value [95% CI] |
|---|---|
| Sensitivity | 0.84 [0.74-0.90] |
| Specificity | 0.79 [0.66-0.88] |
| AUC-ROC | 0.88 [0.82-0.94] |

The scope for a finer-grain distinction between DCM and MMVD was explored from the available data. Following the same procedure as above, the trained KNN classifier provided modest performance metrics, including accuracy 0.61, AUC 0.61 (95% CI: 0.48-0.74), sensitivity 0.59 (95% CI: 0.43-0.74) and specificity 0.62 (95% CI: 0.46-0.76). The limited group sizes in this case (n=37) probably prevented here from fairly capturing the subtleties in miRNA profiles for a neater differentiation between diseased groups, also leading to larger uncertainty in performance metrics.

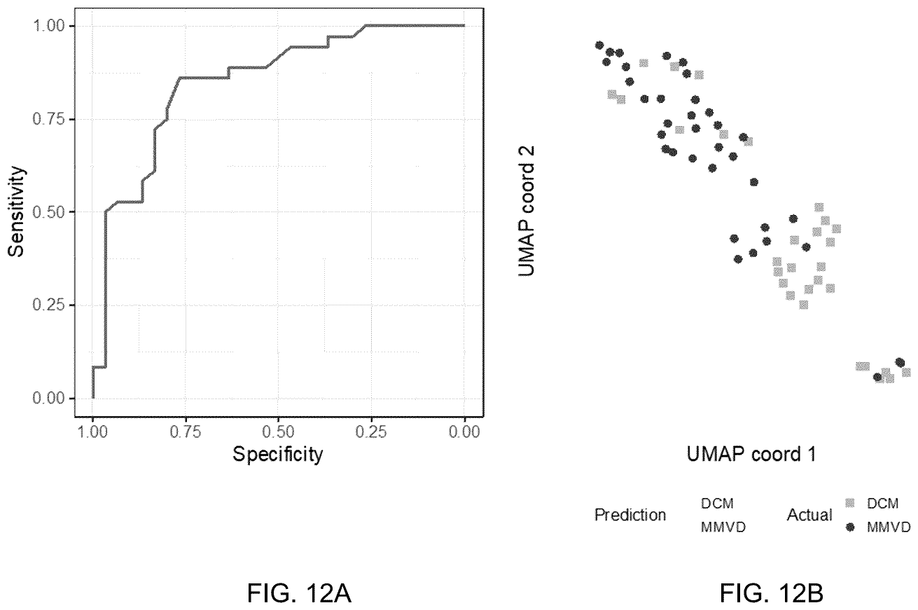

In another study, samples from of MMVD (n=36) and DCM (n-30) were screened using the described microRNA panel and laboratory Fireplex® protocol as shown in Table 11 and FIGS. 12A-12B.

TABLE 11
DCM (30) vs MMVD (36) analysis

Gold standard cardiac evaluation

| | | DCM | MMVD |
|---|---|---|---|
| MicroRNA | DCM | 34.85% | 7.58% |
| cardiac platform | MMVD | 10.61% | 46.97% |

| | Cross-validated value |
|---|---|
| Performance metric | [95% CI] |
| Sensitivity | 0.77 [0.59-0.88] |
| Specificity | 0.86 [0.71-0.94] |
| AUC-ROC | 0.85 [0.75-0.95] |
| Accuracy | 0.82 |

The trained KNN classifier provided good performance metrics, including accuracy 0.82, AUC 0.85 (95% CI: 0.75-0.95), sensitivity 0.77 (95% CI: 0.59-0.88) and specificity 0.86 (95% CI: 0.71-0.94). FIGS. 12A-12B shows the AUC and a UMAP demonstrating distinct separate grouping of the DCM and MMVD miRNA profiles. This data shows the current cardiac miRNA panel provides high resolution to distinguish between DCM and MMVD cases using machine learning algorithms.

### Example 9: MiRNA Profiling with Machine Learning Optimization: Feline HCM

### Materials and Methods

Cardiac panel miRNA profiles from the 2021 datasets (18 controls and 34 HCM cases) were normalised in Fireplex using previously identified control miRNAs. Data were standardized and Yeo Johnson transformed. Eighteen machine learning algorithms were trained with 10-fold cross validation across five repeats (15 models previously used for MMVD plus XGBoost, PLSDA and GLM). Model classification accuracy was assessed using ROC AUC, overall accuracy, sensitivity, and specificity. Variable importance was estimated using individual ROC curve analysis.

### Results

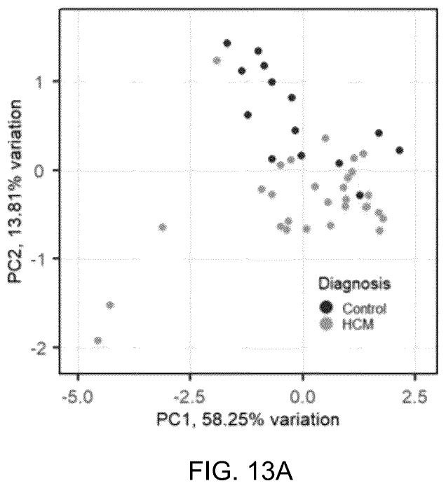

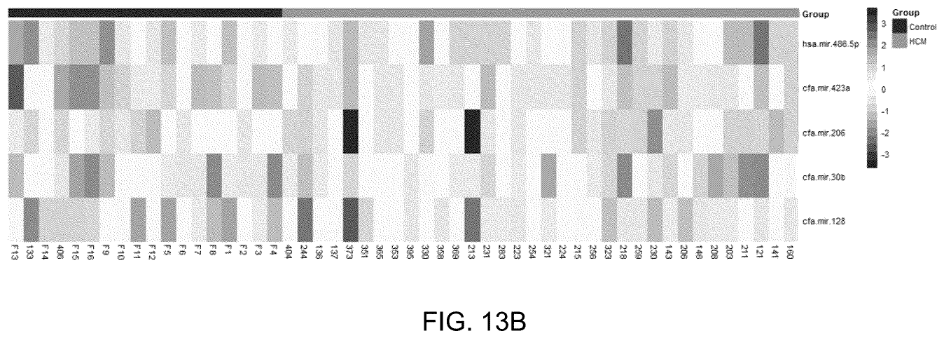

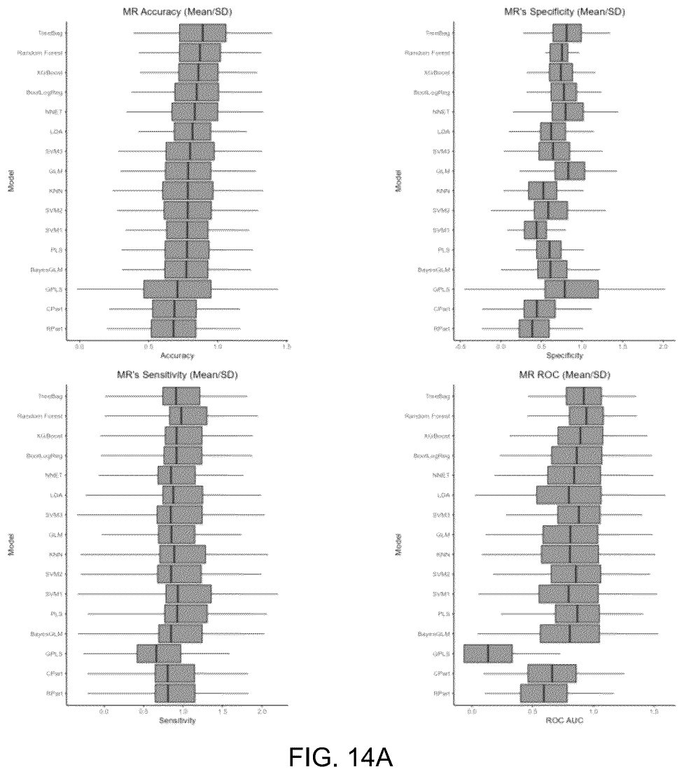

Feline miRNA profiles of controls and HCM samples appear to separate on the second axis of a principal component analysis (PCA) (FIG. 13A). An expression heatmap of the most important variables (AUC>0.6 in individual ROC analyses) show that five miRNAs that appear to differ in expression between controls and HCM samples (FIG. 13B).

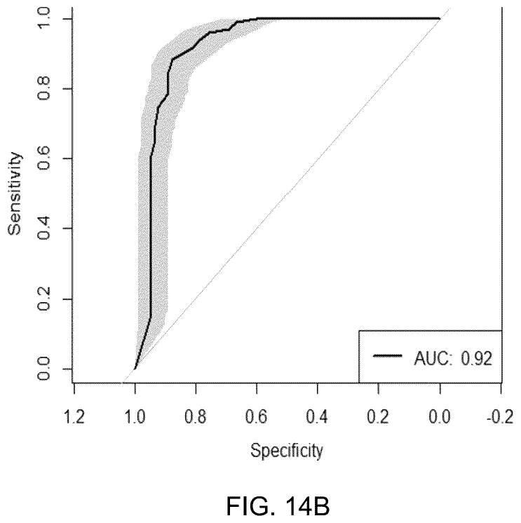

The most accurate model for binary classification (control vs HCM) was Bootstrap Tree Aggregation (TreeBag), with an overall accuracy 87%, 0.92 ROC AUC, sensitivity 0.83 and specificity 0.94 (FIG. 14A). Estimates of the 95% confidence intervals for the ROC AUC using the TreeBag model suggest that it is discriminative for the test data (FIG. 14B).

The current cardiac miRNA panel previously validated on canine MMVD samples can distinguish between control and feline HCM samples in the test data set with high accuracy using a TreeBag model trained using machine learning.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method in a computer-implemented system comprising at least one processor and at least one memory, the at least one memory comprising instructions executed by the at least one processor to cause the at least one processor to implement one or more predictive classification models to assess and differentially diagnose a cardiac disease or conditions in a subject, comprising the steps of:
(a) obtaining a sample from the subject;
(b) determining a level of expression of each of a plurality of miRNA molecules within the sample;
(c) applying the one or more predictive classification models to the expression of each of a plurality of miRNA molecules;
(d) using the predictive classification models to differentially classify the diseased state of the cardiac disease or condition in the subject; and
(e) using the classification of the diseased state of the cardiac disease or condition to predict the disease condition of the subject;
wherein the cardiac condition is myxomatous mitral valve disease (MMVD), mitral regurgitation (MR), dilated cardiomyopathy disease (DCM), or hypertrophic cardiomyopathy (HCM).

2. The method of claim 1, wherein the method further comprises a step of using one or more machine learning algorithms to generate predictive classification models.

3. The method according to claim 1, wherein the one or more predictive classification models compares the level of expression of each miRNA molecule with at least one pre-determined reference level characteristic of a non-diseased subject for each one of the plurality of the miRNA molecules of step (b), wherein a deviation of the level of expression of said miRNA molecules from step (b) in comparison with the at least one reference level allows for the diagnosis and/or prognosis of the disease.

4. The method according to claim 1, wherein the plurality of miRNA molecules from a panel selected from a group consisting of miRNAs having at least 99% sequence identity to SEQ ID NO: 1, 2, 3, 4, 5, 6, 7, 8, 9, 10, 11, 12, 13, 14, 15 or the combination thereof.

5. The method of claim 1, wherein the method comprises the use of a combination of predictive classification models.

6. The method of claim 1, wherein application of the predictive classification models distinguishes non-diseased subjects from diseased subjects with the mitral valve disease or condition, wherein the non-diseased subjects correspond to stage A subjects as classified by the American College of Veterinary Internal Medicine (ACVIM) classification system and the diseased subjects with the mitral valve disease or condition correspond to stages B1, B2, C and D subjects as classified by the ACVIM classification system.

7. The method of claim 1, wherein application of the predictive classification models distinguishes pre-clinical mitral valve diseases or conditions subjects from clinical mitral valve diseases or conditions subjects, wherein the preclinical mitral valve disease or condition subjects correspond to stage B1 and stage B2 subjects as classified by the ACVIM classification system and the clinical mitral valve disease or condition subjects correspond to stage C and stage D subjects as classified by the ACVIM classification system.

8. The method of claim 1, wherein the mitral valve disease or condition is myxomatous mitral valve disease (MMVD) or mitral regurgitation (MR).

9. The method of claim 1, wherein application of the predictive classification models distinguishes non-diseased subjects from diseased subjects with the dilated cardiomyopathy (DCM) disease or condition.

10. The method of claim 1, wherein application of the predictive classification models distinguishes non-diseased subjects from diseased subjects with the hypertrophic cardiomyopathy (HCM) disease or condition.

11. The method of claim 1, wherein the method differentially assesses and diagnoses one cardiac disease from another.

12. The method of claim 1, wherein the method differentially assesses and diagnoses myxomatous mitral valve disease (MMVD) from dilated cardiomyopathy (DCM).

13. The method of claim 1, wherein the subject is a mammal selected from a group of non-human mammals consisting of dogs, cats, and horses.

14. The method of claim 1, wherein the method further comprises the use of at least one normalizer and/or control miRNA molecule.

15. The method of claim 14, wherein the control miRNA molecule is an off-species control miRNA molecule.

16. The method according to claim 14, wherein the at least one normalizer is selected from a group consisting of miRNAs having at least 99% sequence identity to SEQ ID NO: 16, 17, 18, 19, and 20.

17. The method of claim 1, wherein the sample is selected from a group consisting of a tissue or organ sample, blood sample, urine, saliva, milk and cerebrospinal fluid sample.

18. The method of claim 17, wherein the blood sample is selected from the group consisting of serum, plasma, cell-free blood, whole blood and its components, blood derived products or preparations thereof.

19. The method according to claim 1, wherein the miRNAs are cell free miRNAs.

20. A method of selecting a miRNA panel for use in disease assessment and diagnosis of a cardiac disease in a subject comprising the steps of:
(a) obtaining a sample from the subject;
(b) determining a level of expression of each of a plurality of miRNA molecules within the sample, having at least 99% sequence identity to SEQ ID NO: 1, 2, 3, 4, 5, 6, 7, 8, 9, 10, 11, 12, 13, 14, and 15;
(c) using the computer-implemented system of claim 1 comprising at least one processor and at least one memory, the at least one memory comprising instructions executed by the at least one processor to cause the at least one processor to apply machine learning algorithms to generate one or more predictive classification models;
(d) applying the one or more predictive classification models to the expression of each of a plurality of miRNA molecules; and
(e) using the predictive classification models to diagnose the cardiac disease in the subject;
wherein the cardiac condition is myxomatous mitral valve disease (MMVD), mitral regurgitation (MR), dilated cardiomyopathy disease (DCM), or hypertrophic cardiomyopathy (HCM), and
wherein the method differentially assesses and diagnoses a preclinical or clinical stage of the cardiac disease or differentially diagnoses one cardiac disease from another.
